SEQUENCE LISTING (9 sequences)

SEQ ID NO: 1 - protein, 579 aa, Synthetic construct
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLSQSYINDKGKEVLVLWGIHHPSTTADQQSLYQNADAYVFVGTSRYSKKFKPEIAIRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYGSGYIPEAPRDGQAYVRKDGEWVLLSTFLGLNDIFEAQKIEWHEGHHHHHH

SEQ ID NO: 2 - protein, 573 aa, Synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGSGLNDIFEAQKIEWHEGHHHHHH

SEQ ID NO: 3 - protein, 566 aa, Synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFONIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 4 - protein, 566 aa, Synthetic construct
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQXLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 5 - protein, 566 aa, Synthetic construct
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLSQSYINDKGKEVLVLWGIHHPSTTADQQSLYQNADAYVFVGTSRYSKKFKPEIAIRPKVRDQEGRMNYYWTLVEPGDKITFEATGNLVVPRYAFTMERNAGSGIIISDTPVHDCNTTCQTPEGAINTSLPFONVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 6 - protein, 566 aa, Synthetic construct
MKAILVVLLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFONVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 7 - protein, 566 aa, Synthetic construct
MKARLLVLLCALAATDADTICIGYHANNSTDTVDTVLEKNVTVTHSVNLLEDSHNGKLCKLKGIAPLQLGKCNIAGWLLGNPECESLLSARSWSYIVETPNSENGTCYPGDFIDYEELREQLSSVSSFERFEIFPKESSWPNHNTTKGVTAACSHAGKSSFYRNLLWLTKKGGSYPKLSKSYVNNKGKEVLVLWGVHHPSTSTDQQSLYQNENAYVSVVSSNYNRRFTPEIAERPKVRGQAGRMNYYWTLLEPGDTIIFEATGNLIAPWYAFALSRGSGSGIITSNASMHECNTKCQTPQAGRMNYYWTLLEPGDTIIFEATGNLIAPWYAFALSRGSGSGIITSNASMHECNTKCQTPQMIDGWYGYHHQNEQGSGYAADQKSTQNAINGITNKVNSVIEKMNTQFTAVGKEFNNLEKRMENLNKKVDDGFLDIWTYNAELLVLLENERTLDFHDSNVKNLYEKVKSQLRNNAKEIGNGCFEFYHKCDNECMESVKNGTYDYPKYSEESKLNREKIDGVKLESMGVYQILAIYSTVASSLVLLVSLGAISFWMCSNGSLQCRICI

SEQ ID NO: 8 - protein, 565 aa, Synthetic construct
MEARLLVLLCAFAATNADTICIGYHANNSTDTVDTVLEKNVTVTHSVNLLEDSHNGKLCLLKGIAPLOLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYANNKEKEVLVLWGVHHPPNIGDQRALYHTENAYVSVVSSHYSRKFTPEIAKRPKVRDQEGRINYYWTLLEPGDTIIFEANGNLIAPRYAFALSRGFGSGIITSNAPMDECDAKCQTPQGAINSSLPFONVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 9 - protein, 565 aa, Synthetic construct
MKVKLLVLLCTFTATYADTICIGYHANNSTOTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGNOKALYHTENAYVSVVSSHYSRKFTPEIAKRPKVRDQEGRINYYWTLLEPGDTIFEANGNLIAPRYAFALSRGFGSGIINSNAPMDKCDAKCQTPQGAINSSLPFONVHPVTIGECPKYVRSAKLRMVTGLRNIPSIQSRGLFGAIAGFIEGGWTGMVDGWYGYHHQONEQGSGYAADQKSTONAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

(19) United States
(12) Patent Application Publication
(10) Pub. No.: US 2024/0058434 A1
(43) Pub. Date: Feb. 22, 2024

(54) NANOEMULSION UNIVERSAL INFLUENZA VACCINE

(71) Applicant: BlueWillow Biologics, Inc., Ann Arbor, MI (US)

(72) Inventors: Shyamala Ganeson, Novi, MI (US); Vira Bitko, Ann Arbor, MI (US); Ali Fattom, Plymouth, MI (US)

(73) Assignee: BlueWillow Biologics, Inc., Ann Arbor, MI (US)

(21) Appl. No.: 18/133,238

(22) Filed: Apr. 11, 2023

Related U.S. Application Data
(60) Provisional application No. 63/330,257, filed on Apr. 12, 2022.

(51) Int. Cl. A61K 39/145; A61P 31/16

(52) U.S. Cl. CPC A61K 39/145; A61P 31/16; A61K 2039/55566

(57) ABSTRACT

The present invention relates to methods for inducing a broadly reactive immune response to multiple strains of influenza in a subject comprising intranasally administering a nanoemulsion vaccine composition comprising a computationally optimized influenza immunogen or protein.

### CROSS-REFERENCE TO RELATED APPLICATION

This application claims the benefit under 35 U.S.C. § 119(e) of U.S. Provisional Patent Application Ser. No. 63/330,257, filed Apr. 12, 2022, the entire contents of which is incorporated herein by reference.

### FIELD OF INVENTION

The present invention provides intranasal nanoemulsion universal influenza vaccines, which provide protection against multiple strains of influenza, and methods of using the same.

### SEQUENCE LISTING

The specification further incorporates by reference the Sequence Listing submitted herewith. The Sequence Listing is identified as 038491_0346_Sequence_Listing.xml, and is 14 bytes in size and was created on Jun. 19, 2023.

### BACKGROUND OF THE INVENTION

Influenza is a serious public health threat, routinely killing hundreds of thousands of people worldwide each year, and millions during pandemics. The World Health Organization (WHO) estimates annual influenza epidemics cause 2-5 million severe cases and 250,000 to 500,000 deaths. Influenza disease symptoms range from mild fever, sore throat, coughing, nasal discharge, headache, and myalgia to more severe cases that can lead to the development of bronchitis or pneumonia. There are four types of influenza viruses, including A, B, C and D, of which types A and B cause seasonal epidemics in humans. The current circulating influenza A subtypes are H1N1 and H3N2, and these subtypes have caused three of the four influenza pandemics in the last century, including the 1918 H1N1 “Spanish flu” pandemic, the 1968 H3N2 “Hong Kong flu” pandemic, and the 2009 H1N1 “Swine flu” pandemic. H3N2 influenza viruses evolve more rapidly than H1N1 viruses, which results in more frequent updates to the H3N2 strain used in seasonal influenza vaccines, and the WHO has recommended 28 vaccine strain changes since their introduction.

Influenza A virus is the pathogen associated with all known flu pandemics and is currently the most virulent form of the virus. A number of distinct serotypes have been isolated, including H1N1, H1N2, H2N2, H3N1, H3N2, H3N8, H5N1, H5N2, H5N3, H5N8, H5N9, H7N1, H7N2, H7N3, H7N4, H7N7, H9N2 and H10N7. These serotypes are classified according to two viral surface proteins, hemagglutinin (H or HA) and neuroaminidase (N or NA). Within these serotypes, isolates are further characterized by a standard nomenclature specifying virus type, geographical location where first isolated, sequential number of isolation, year of isolation, and HA and NA subtype. For instance, one such isolate is A/Wisconsin/67/2005 (H3N2).

Influenza virus is a global respiratory pathogen that has been circulating in humans for hundreds of years. Due to the highly variable and mutable nature of influenza antigens, developing a vaccine useful against multiple influenza strains has proven difficult. At present, the annual flu vaccine must be reformulated and readministered each year in anticipation of the serotypes of the virus predicted to be prevalent in a population each flu season, and is therefore considered a “seasonal” vaccine. Typically, the most common human flu vaccine is a combination of two influenza A subtypes and one influenza B strain.

U.S. Pat. Nos. 7,314,624; 9,144,606 and 10,525,121 describe nanoemulsion influenza vaccines. However, these references do not teach the ability to induce a protective immune response against multiple influenza strains.

While many successful vaccines against Influenza have been produced, influenza vaccines are typically directed only to a single influenza strain. Due to the fast-evolving nature of Influenza viruses and the sheer number of distinct circulating strains at any one given time, single-strain strategies to vaccine manufacturing may be costly and functionally insufficient.

Thus, there is a need for a “universal” influenza vaccine which provides protection against multiple influenza strains. The present disclosure satisfies this need.

### SUMMARY OF THE INVENTION

Provided herein is a nanoemulsion influenza vaccine formulated for intranasal administration and comprising: (a) at least one computationally optimized broadly reactive antigen (COBRA) influenza antigen; (b) a nanoemulsion vaccine adjuvant comprising droplets having an average diameter of less than about 1000 nm. The nanoemulsion comprises: (i) an aqueous phase; (ii) at least one pharmaceutically acceptable oil; (iii) a combination of at least one cationic surfactant and at least one non-ionic surfactant; and (iv) at least one pharmaceutically organic solvent. Further, the COBRA influenza antigen is associated with the droplets of the nanoemulsion vaccine adjuvant. Finally, upon administration the vaccine produces a protective immune response against more than one influenza viral strain.

In some embodiments, administration of a nanoemulsion influenza vaccine to a subject in need of the vaccine induces an immune response in the subject comprising the production of antibodies capable of recognizing at least two different hemagglutinins (HA) proteins. In some embodiments, an immune response in the subject comprises the production of antibodies capable of recognizing HA proteins from at least 2, at least 3, at least 4, at least 5, at least 6, at least 7, at least 8, at least 9, at least 10, at least 11, at least 12, at least 13, at least 14, at least 15, at least 16, at least 17, or 18 different HA proteins from influenza A subtypes. In some embodiments, an immune response in the subject comprises the production of antibodies capable of recognizing HA proteins from influenza B/Yamagata and/or B/Victoria. In some embodiments, administration of a nanoemulsion influenza vaccine to a subject in need of the vaccine induces an immune response in the subject comprising the production of antibodies capable of recognizing HA proteins from all influenza A and/or B strains identified since 1933. In some embodiments, an immune response in the subject comprises the production of antibodies capable of recognizing HA proteins from at least one influenza A virus and at least one influenza B virus. In other aspects, an immune response in the subject comprises the production of antibodies capable of recognizing HA proteins from at least one influenza A virus, at least one influenza B virus, and at least one influenza C virus.

In some embodiments, administration of a nanoemulsion influenza vaccine to a subject in need of the vaccine induces an immune response in the subject comprising the production of antibodies capable of recognizing at least two different neuramidase (NA) proteins from influenza A, B, or C. In some embodiments, administration of a nanoemulsion influenza vaccine to a subject in need of the vaccine induces an immune response in the subject comprising the production of antibodies capable of recognizing NA proteins from all influenza A and/or B strains identified since 1933. In some embodiments, an immune response in the subject comprises the production of antibodies capable of recognizing at least 2, at least 3, at least 4, at least 5, at least 6, at least 7, at least 8, at least 9, at least 10, or 11 different NA subtypes of influenza A. Further, in some embodiments, an immune response in the subject comprises the production of antibodies capable of recognizing different NA subtypes from B/Yamagata and/or B/Victoria influenza.

In some embodiments, administration of a nanoemulsion influenza vaccine to a subject in need of the vaccine induces an immune response in the subject comprising a Th1 immune response, a Th2 immune response, a Th17 immune response, or any combination thereof. In some embodiments, administration of a nanoemulsion influenza vaccine to a subject in need of the vaccine induces a balanced and protective Th1/Th2 immune response in the subject. In some embodiments, administration of a nanoemulsion influenza vaccine to a subject in need of the vaccine induces a systemic, mucosal, and cell-mediated immune response in the subject. In some embodiments, administration of a nanoemulsion influenza vaccine to a subject in need of the vaccine induces a robust systemic, mucosal, and cell-mediated immune response with minimal inflammation. In some embodiments, administration of a nanoemulsion influenza vaccine to a subject in need of the vaccine induces protection at the site of mucosal infection for the subject. In some embodiments, administration of a nanoemulsion influenza vaccine to a subject in need of the vaccine induces IL17 and IgA in the subject to provide mucosal protection.

In some embodiments, a COBRA influenza antigen comprises recombinant hemagglutinins (rHAs) based upon the amino acid sequence of at least one influenza viral strain. In some embodiments, a COBRA influenza antigen is based upon one or more amino acid sequences of influenza A and/or B viruses identified from 1900 to 2022. In some embodiments, a COBRA HA sequence is based upon one or more HA amino acid sequences from clade 2 H5N1 human infections. In some embodiments, a COBRA NA sequence is based upon one or more NA amino acid sequences from clade 2 H5N1 human infections.

In some embodiments, a COBRA optimized influenza antigen is based upon an amino acid sequence from one or more of the following influenza viral strains: (a) H1, a recombinant immunogenic variant of H1, or an immunogenic fragment of H1; (b) H2, a recombinant immunogenic variant of H2, or an immunogenic fragment of H2; (c) H3, a recombinant immunogenic variant of H3, or an immunogenic fragment of H3; (d) H5, a recombinant immunogenic variant of H5, or an immunogenic fragment of H5; (e) H7, a recombinant immunogenic variant of H7, or an immunogenic fragment of H7; (f) H9, a recombinant immunogenic variant of H9, or an immunogenic fragment of H9; (g) N1, a recombinant immunogenic variant of N1, or an immunogenic fragment of N1; (h) N2, a recombinant immunogenic variant of N2, or an immunogenic fragment of N2; (i) N3, a recombinant immunogenic variant of N3, or an immunogenic fragment of N3; ( ) N7, a recombinant immunogenic variant of N7, or an immunogenic fragment of N7; (k) a seasonal influenza strain, a recombinant immunogenic variant of a seasonal influenza strain, or an immunogenic fragment of a seasonal influenza strain; (1) a pandemic influenza strain, a recombinant immunogenic variant of a pandemic influenza strain, or an immunogenic fragment of a pandemic influenza strain; (m) an influenza A virus strain, a recombinant immunogenic variant of an influenza A virus strain, or an immunogenic fragment of an influenza A virus strain; (n) an influenza B virus strain, a recombinant immunogenic variant of an influenza B virus strain, or an immunogenic fragment of an influenza B virus strain; (o) an influenza C virus strain, a recombinant immunogenic variant of an influenza C virus strain, or an immunogenic fragment of an influenza C virus strain; (p) A/New Caledonia/20/99 lineage; (q) A/Fujian/411/2002 lineage; (r) A/Kumamoto/102/2002 lineage; (s) A/Wyoming/3/2003 lineage; (t) A/Wellington/1/2004 lineage; (u) A/California/7/2004 lineage; (v) A/New York/55/2004 lineage; (w) A/Solomon Islands/3/2006 lineage; (x) A/Wisconsin/67/2005 lineage; (y)A/Hiroshima/52/2005 lineage; (z) A/Brisbane/10/2007 lineage; (aa) B/Hong Kong/330/2001 lineage; (bb) B/Shandong/7/97 lineage; (cc) B/Hong Kong/1434/2002 lineage; (dd) B/Brisbane/32/2002 lineage; (ee) B/Shanghai/361/2002 lineage; (ff) B/Jiangsu/10/2003 lineage; (gg) B/Jilin/20/2003 lineage; (hh) B/Malaysia/2506/2004 lineage; (ii) B/Florida/4/2006 lineage, (jj) B/Victoria/2/87 lineage, (kk) B/Yamagata/16/88 lineage, (ll) C/Aichi/1/99 lineage, (mm)C/Sao Paulo/378/82 lineage, (nn) C/Yamagata/26/81 lineage, (oo) C/Aichi/1/81 lineage, (pp) C/Aomori/74 lineage; (qq) C/Mississippi/80 lineage; (rr) any new strain or subtype that may arise due to antigenic drift and/or mutation and (aa) any combination thereof.

In some embodiments, a nanoemulsion vaccine: (a) is not systemically toxic to the subject; (b) produces minimal or no inflammation upon administration; or (c) any combination thereof.

In some embodiments, nanoemulsion vaccine adjuvant droplets have an average diameter selected from the group consisting less than about 1000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, greater than about 50 nm, greater than about 70 nm, greater than about 125 nm, and any combination thereof. In some embodiments, nanoemulsion vaccine vaccine adjuvant droplets have an average diameter greater than about 125 nm and less than about 600 nm.

In some embodiments, an organic solvent: (a) is selected from the group consisting of a C₁-C₁₂ alcohol, diol, triol, dialkyl phosphate, tri-alkyl phosphate, and combinations thereof; (b) is an alcohol selected from the group consisting of a nonpolar solvent, a polar solvent, a protic solvent, an aprotic solvent, semi-synthetic derivatives thereof, and combinations thereof, or (c) any combination thereof.

In some embodiments, an oil is: (a) any cosmetically or pharmaceutically acceptable oil; (b) non-volatile; (c) selected from the group consisting of animal oil, vegetable oil, natural oil, synthetic oil, hydrocarbon oils, silicone oils, and semi-synthetic derivatives thereof; (d) selected from the group consisting of mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (Simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and combinations thereof; or (d) any combination thereof.

In some embodiments, a non-ionic surfactant is selected from the group consisting of nonoxynol-9, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), Brij®35, Brij 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, Heptaethylene glycol monodecyl ether, Heptaethylene glycol monotetradecyl ether, Heptaethylene glycol monododecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Methyl-6-O-(N-heptylcarbamoyl)-alpha-D-glucopyranoside, Nonaethylene glycol monododecyl ether, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Tergitol Type 15-S-12, Tergitol Type 15-S-30, Tergitol Type 15-S-5, Tergitol Type 15-S-7, Tergitol Type 15-S-9, Tergitol Type NP-10, Tergitol Type NP-4, Tergitol Type NP-40, Tergitol Type NP-7, Tergitol Type NP-9, Tergitol Type TMN-10, Tergitol Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton X-114, Triton X-165, Triton X-305, Triton X-405, Triton X-45, Triton X-705-70, a polysorbate, polysorbate 20, polysorbate 21, polysorbate 40, polysorbate 60, polysorbate 61, polysorbate 65, polysorbate 80, polysorbate 81, polysorbate 85, Tyloxapol, n-Undecyl beta-D-glucopyranoside, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, Poloxamer 182, Dibenzoate, semi-synthetic derivatives thereof, and combinations thereof. In some embodiments, a non-ionic surfactant is a polysorbate. In some embodiments, a polysorbate is polysorbate 20 or polysorbate 80. In some embodiments, a nonionic surfactant is present in a vaccine at about 0.01% to about 10%, about 0.05% to about 10%, about 0.05% to about 7.0%, about 0.1% to about 7%, about 0.1% to about 3%, or about 0.5% to about 4% (w/w).

In some embodiments, a cationic surfactant is selected from the group consisting of a quarternary ammonium compound, an alkyl trimethyl ammonium chloride compound, a dialkyl dimethyl ammonium chloride compound, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Cetylpyridinium chloride, Dimethyldioctadecylammonium bromide, Dodecylethyldimethylammonium bromide, Dodecyltrimethylammonium bromide, Ethylhexadecyldimethylammonium bromide, Girard's reagent T, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl didecyl dimethyl ammonium chloride, Alkyl dimethyl benzyl ammonium chloride (C12-16), Alkyl dimethyl benzyl ammonium chloride (C12-18), dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis (2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof. In some embodiments, a cationic surfactant which is cetylpyridinium chloride.

In some embodiments, a concentration of a cationic surfactant is less than about 5.0% and greater than about 0.001%, about 0.05% to about 2%, or about 0.01% to about 2% (w/w). In some embodiments, a concentration of a cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.001%, greater than about 0.002%, greater than about 0.003%, greater than about 0.004%, greater than about 0.005%, greater than about 0.006%, greater than about 0.007%, greater than about 0.008%, greater than about 0.009%, and greater than about 0.010% (w/w).

In some embodiments, a nanoemulsion vaccine adjuvant comprises: (a) an aqueous phase; (b) about 1% oil to about 80% (w/w) of at least one pharmaceutically acceptable oil; (c) about 0.1% organic solvent to about 50% (w/w) organic solvent; (d) about 0.001% surfactant to about 10% (w/w) surfactant; (e) less than about 5.0% and greater than about 0.001% (w/w) of at least one cationic surfactant; and (f) about 0.01% to about 10% (w/w) of at least one non-ionic surfactant. In some embodiments, a vaccine may include at least one preservative. A preservative may be selected from the group consisting of cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, Other suitable preservatives include, but are not limited to, benzyl alcohol, chlorhexidine (bis (p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens), and combinations thereof.

In some embodiments, a vaccine may include at least one pH adjuster. A pH adjuster may be selected from the group consisting of diethanolamine, lactic acid, monoethanolamine, triethylanolamine, sodium hydroxide, sodium phosphate, semi-synthetic derivatives thereof, and combinations thereof.

In some embodiments, a vaccine may include at least one buffer. A buffer may be selected from the group consisting of 2-Amino-2-methyl-1,3-propanediol, 2-Amino-2-methyl-1-propanol, L-(+)-Tartaric acid, ACES, ADA, Acetic acid, Ammonium acetate solution, Ammonium bicarbonate, Ammonium citrate dibasic, Ammonium formate, Ammonium oxalate monohydrate, Ammonium phosphate dibasic, Ammonium phosphate monobasic, Ammonium sodium phosphate dibasic tetrahydrate, Ammonium sulfate solution, Ammonium tartrate dibasic, BES buffered saline, BES, BICINE, BIS-TRIS, Bicarbonate buffer solution, Boric acid, CAPS, CHES, Calcium acetate hydrate, Calcium carbonate, Calcium citrate tribasic tetrahydrate, Citrate Concentrated Solution, Citric acid, hydrous, Diethanolamine, EPPS, Ethylenediaminetetraacetic acid disodium salt dihydrate, Formic acid solution, Gly-Gly-Gly, Gly-Gly, Glycine, HEPES, Imidazole, Lipoprotein Refolding Buffer, Lithium acetate dihydrate, Lithium citrate tribasic tetrahydrate, MES hydrate, MES monohydrate, MES solution, MOPS, Magnesium acetate solution, Magnesium acetate tetrahydrate, Magnesium citrate tribasic nonahydrate, Magnesium formate solution, Magnesium phosphate dibasic trihydrate, Oxalic acid dihydrate, PIPES, Phosphate buffered saline, Piperazine, Potassium D-tartrate monobasic, Potassium acetate, Potassium bicarbonate, Potassium carbonate, Potassium chloride, Potassium citrate monobasic, Potassium citrate tribasic solution, Potassium formate, Potassium oxalate monohydrate, Potassium phosphate dibasic, Potassium phosphate dibasic, for molecular biology, anhydrous, Potassium phosphate monobasic, Potassium phosphate monobasic, Potassium phosphate tribasic monohydrate, Potassium phthalate monobasic, Potassium sodium tartrate, Potassium sodium tartrate tetrahydrate, Potassium tetraborate tetrahydrate, Potassium tetraoxalate dihydrate, Propionic acid, STE buffer, STET buffer, Sodium 5,5-diethylbarbiturate, Sodium acetate, Sodium acetate trihydrate, Sodium bicarbonate, Sodium bitartrate monohydrate, Sodium carbonate decahydrate, Sodium carbonate, Sodium citrate monobasic, Sodium citrate tribasic dihydrate, Sodium formate solution, Sodium oxalate, Sodium phosphate dibasic dihydrate, Sodium phosphate dibasic dodecahydrate, Sodium phosphate dibasic solution, Sodium phosphate monobasic dihydrate, Sodium phosphate monobasic monohydrate, Sodium phosphate monobasic solution, Sodium pyrophosphate dibasic, Sodium pyrophosphate tetrabasic decahydrate, Sodium tartrate dibasic dihydrate, Sodium tartrate dibasic solution, Sodium tetraborate decahydrate, TAPS, TES, TM buffer solution, TNT buffer solution, TRIS Glycine buffer, TRIS acetate—EDTA buffer solution, TRIS buffered saline, TRIS glycine SDS buffer solution, TRIS phosphate-EDTA buffer solution, Tricine, Triethanolamine, Triethylamine, Triethylammonium acetate buffer, Triethylammonium phosphate solution, Trimethylammonium acetate solution, Trimethylammonium phosphate solution, Tris-EDTA buffer solution, Trizma® acetate, Trizma® base, Trizma® carbonate, Trizma® hydrochloride, Trizma® maleate, or any combination thereof.

In some embodiments, an aqueous phase of the vaccine is present in Phosphate Buffered Saline (PBS).

In some embodiments, a vaccine provided herein is stable at about 40° C. and about 75% relative humidity for a time period selected from the group consisting of up to about 2 days, up to about 1 week, up to about 2 weeks, up to about 1 month, up to about 3 months, up to about 6 months, up to about 12 months, up to about 18 months, up to about 2 years, up to about 2.5 years, and up to about 3 years. In some embodiments, a vaccine provided herein is stable at about 25° C. and about 60% relative humidity for a time period selected from the group consisting of up to about 2 days, up to about 1 week, up to about 2 weeks, up to about 1 month, up to about 3 months, up to about 6 months, up to about 12 months, up to about 18 months, up to about 2 years, up to about 2.5 years, up to about 3 years, up to about 3.5 years, up to about 4 years, up to about 4.5 years, and up to about 5 years. In some embodiments, a vaccine provided herein is stable at about 4° C. for a time period selected from the group consisting of up to about 3 months, up to about 6 months, up to about 12 months, up to about 18 months, up to about 2 years, up to about 2.5 years, up to about 3 years, up to about 3.5 years, up to about 4 years, up to about 4.5 years, up to about 5 years, up to about 5.5 years, up to about 6 years, up to about 6.5 years, and up to about 7 years. In some embodiments, a vaccine provided herein is stable at about −20° C. for a time period selected from the group consisting of up to about 3 months, up to about 6 months, up to about 12 months, up to about 18 months, up to about 2 years, up to about 2.5 years, up to about 3 years, up to about 3.5 years, up to about 4 years, up to about 4.5 years, up to about 5 years, up to about 5.5 years, up to about 6 years, up to about 6.5 years, and up to about 7 years.

In some embodiments, a COBRA influenza antigen is a polypeptide comprising an amino acid sequence that is at least 80%, at least 85%, at least 90%, at least 95%, at least 96%, at least 97%, at least 98%, at least 99%, or 100% identical to SEQ ID NO: 1.

In some embodiments, an influenza antigen is a polypeptide comprising an amino acid sequence that is at least 80%, at least 85%, at least 90%, at least 95%, at least 96%, at least 97%, at least 98%, at least 99%, or 100% identical to SEQ ID NO: 2.

In some embodiments, a vaccine provided herein is formulated for intranasal administration via a nasal spray. In some embodiments, a vaccine provided herein is formulated for intranasal administration via a nasal dropper. In some embodiments, a vaccine provided herein is formulated into a dosage form selected from the group consisting of a liquid dispersion, gel, aerosol, nasal aerosol, and suspensions.

In some embodiments, a vaccine provided herein is antigen sparing, meaning that per dose the vaccine comprises less antigen as compared to a commercial influenza vaccine, influenza vaccine, or a pandemic influenza vaccine.

In some embodiments, a vaccine comprises about 0.001 μg to about 150 μg of each COBRA optimized influenza antigen. In some embodiments, a vaccine comprises about 100 μg to about 150 μg COBRA optimized influenza antigen, per dose.

In some embodiments, a vaccine comprises more than one COBRA optimized influenza antigen.

Provided herein is a kit comprising: (a) a vaccine; (b) a device for nasal administration; and optionally (c) instructions for administration of the same.

Provided herein is a method for inducing an immune response to more than one influenza viral strain in a subject comprising administering to a subject a vaccine, wherein upon administration the vaccine produces a protective immune response against more than one influenza viral strain. In some embodiments, a method for inducing an immune response to more than one influenza viral strain in a subject includes administering sequentially or simultaneously administering to a subject: (a) at least one computationally optimized broadly reactive antigen (COBRA) influenza antigen; and (b) a nanoemulsion vaccine adjuvant comprising droplets having an average diameter of less than about 1000 nm. The nanoemulsion comprises: (i) an aqueous phase; (ii) at least one pharmaceutically acceptable oil; (iii) a combination of at least one cationic surfactant and at least one non-ionic surfactant; and (iv) at least one pharmaceutically organic solvent. Further, the COBRA influenza antigen is associated with the droplets of the nanoemulsion vaccine adjuvant, and upon administration the vaccine produces a protective immune response against more than one influenza viral strain. In some embodiments of the methods, either (a) or (b) is administered first for sequential administration. In some embodiments, a subject produces a protective immune response against more than one influenza viral strain after at least a single administration of the vaccine. In some embodiments, a subject undergoes seroconversion against more then one influenza strain after at least a single administration of the vaccine.

In some embodiments of the methods provided herein, a vaccine generates a cross-reactive immune and/or antibody response against more than one viral strain. In some embodiments of methods provided herein, a vaccine generates a cross-neutralizing immune and/or antibody response against more than one viral strain. In some embodiments of methods provided herein, a vaccine generates a broadly reactive, functional immune and/or antibody response against more than one viral strain. In some embodiments of the methods provided herein, a vaccine elicits an immune response against two or more HA or NA subtypes or any other immunogenic fragments or recombinant influenza proteins selected from the group consisting of: (a) H1, a recombinant immunogenic variant of H1, or an immunogenic fragment of H1; (b) H2, a recombinant immunogenic variant of H2, or an immunogenic fragment of H2; (c) H3, a recombinant immunogenic variant of H3, or an immunogenic fragment of H3; (d) H5, a recombinant immunogenic variant of H5, or an immunogenic fragment of H5; (e) H7, a recombinant immunogenic variant of H7, or an immunogenic fragment of H7; (f) H9, a recombinant immunogenic variant of H9, or an immunogenic fragment of H9; (g) N1, a recombinant immunogenic variant of N1, or an immunogenic fragment of N1; (h) N2, a recombinant immunogenic variant of N2, or an immunogenic fragment of N2; (i) N3, a recombinant immunogenic variant of N3, or an immunogenic fragment of N3; (j) N7, a recombinant immunogenic variant of N7, or an immunogenic fragment of N7; (k) a seasonal influenza strain, a recombinant immunogenic variant of a seasonal influenza strain, or an immunogenic fragment of a seasonal influenza strain; (1) a pandemic influenza strain, a recombinant immunogenic variant of a pandemic influenza strain, or an immunogenic fragment of a pandemic influenza strain; (m) an influenza A virus strain, a recombinant immunogenic variant of an influenza A virus strain, or an immunogenic fragment of an influenza A virus strain; (n) an influenza B virus strain, a recombinant immunogenic variant of an influenza B virus strain, or an immunogenic fragment of an influenza B virus strain; (o) an influenza C virus strain, a recombinant immunogenic variant of an influenza C virus strain, or an immunogenic fragment of an influenza C virus strain; (p) A/New Caledonia/20/99 lineage; (q) A/Fujian/411/2002 lineage; (r) A/Kumamoto/102/2002 lineage; (s) A/Wyoming/3/2003 lineage; (t) A/Wellington/1/2004 lineage; (u) A/California/7/2004 lineage; (v) A/New York/55/2004 lineage; (w) A/Solomon Islands/3/2006 lineage; (x) A/Wisconsin/67/2005 lineage; (y)A/Hiroshima/52/2005 lineage; (z) A/Brisbane/10/2007 lineage; (aa) B/Hong Kong/330/2001 lineage; (bb) B/Shandong/7/97 lineage; (cc) B/Hong Kong/1434/2002 lineage; (dd) B/Brisbane/32/2002 lineage; (ee) B/Shanghai/361/2002 lineage; (ff) B/Jiangsu/10/2003 lineage; (gg) B/Jilin/20/2003 lineage; (hh) B/Malaysia/2506/2004 lineage; (ii) B/Florida/4/2006 lineage, (jj) B/Victoria/2/87 lineage, (kk) B/Yamagata/16/88 lineage, (ll) C/Aichi/1/99 lineage, (mm) C/Sao Paulo/378/82 lineage, (nn) C/Yamagata/26/81 lineage, (oo) C/Aichi/1/81 lineage, (pp) C/Aomori/74 lineage, (qq) C/Mississippi/80 lineage, (rr) any new strain or subtype that may arise due to antigenic drift and/or mutation, and (aa) any combination thereof.

In some embodiments of the methods provided herein, a subject can be selected from the group consisting of adults, elderly subjects, juvenile subjects, infants, high risk subjects, pregnant women, and immuno-compromised subjects.

In some embodiments, at least a single administration of the vaccine is given at a minimum annually to address seasonal influenza, pandemic influenza, or a combination thereof. In some embodiments, one or more administrations of the vaccine are given to the subject to provide sustained protection.

In some embodiments of the methods provided herein, an immune response in the subject comprises the production of antibodies capable of recognizing at least two different HA proteins. In some embodiments, an immune response in the subject comprises the production of antibodies capable of recognizing HA proteins from all influenza A and/or B strains identified since 1933. In some embodiments, an immune response in the subject comprises the production of antibodies capable of recognizing HA proteins from at least one influenza A virus and at least one influenza B virus. In other aspects, an immune response in the subject comprises the production of antibodies capable of recognizing HA proteins from at least one influenza A virus, at least one influenza B virus, and optionally at least one influenza C virus.

In some embodiments, an immune response in the subject comprises the production of antibodies capable of recognizing HA proteins from at least 2, at least 3, at least 4, at least 5, at least 6, at least 7, at least 8, at least 9, at least 10, at least 11, at least 12, at least 13, at least 14, at least 15, at least 16, at least 17, or 18 different HA proteins from influenza A subtypes. In some embodiments, an immune response in the subject comprises the production of antibodies capable of recognizing HA proteins from influenza B/Yamagata and/or B/Victoria.

In some embodiments, an immune response in the subject comprises the production of antibodies capable of recognizing at least two different NA proteins from influenza A, B, or C. In some embodiments, an immune response in the subject comprises the production of antibodies capable of recognizing NA proteins from all influenza A and/or B strains identified since 1933. In some embodiments, an immune response in the subject comprises the production of antibodies capable of recognizing at least 2, at least 3, at least 4, at least 5, at least 6, at least 7, at least 8, at least 9, at least 10, or 11 different NA subtypes of influenza A. Further, in some embodiments, an immune response in the subject comprises the production of antibodies capable of recognizing different NA subtypes from B/Yamagata and/or B/Victoria influenza.

In some embodiments, an immune response in the subject comprises a Th1 immune response, a Th2 immune response, a Th17 immune response, or any combination thereof. In some embodiments, an immune response in the subject comprises a balanced and protective Th1/Th2 immune response. In some embodiments, an immune response in the subject comprises a systemic, mucosal, and cell-mediated immune response. In some embodiments, an immune response in the subject comprises a robust systemic, mucosal, and cell-mediated immune response with minimal inflammation. In some embodiments, an immune response in the subject comprises protection at the site of mucosal infection. In some embodiments, an immune response in the subject comprises inducement of IL17 and IgA to provide mucosal protection.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 shows a diagrammatic illustration of the experimental design and timecourse for the experiments described in the Examples section. FIG. 1A shows the experimental timecourse for vaccinating, challenging, and isolating biological samples from mice treated with three doses of the vaccine formulation. FIG. 1B shows the experimental timecourse for vaccinating, challenging, and isolating biological samples from mice treated with two doses of the vaccine formulation. FIG. 1C shows the experimental timecourse for vaccinating, challenging, and isolating biological samples from mice treated with one dose of the vaccine formulation

FIG. 2 shows the hemagglutination inhibition (HAI) titer against (A) Influenza strain CA09), (B) Influenza strain Bris 18, and (C) Influenza strain Gaundong-Maonon 19 of serum samples isolated from mice administered the vaccine formulation under various treatment conditions

FIG. 3 shows the hemagglutination inhibition (HAI) titer against Influenza strain TX/12 of serum samples isolated from mice administered the vaccine formulation under various treatment conditions.

FIG. 4 shows data illustrating weightloss (A) and survival (B) of mice challenged with Influenza strain Bris 18, under various vaccination conditions.

FIG. 5 shows Bris 18 viral titer data from lung tissue samples isolated from mice challenged with Bris 18, under various vaccination conditions.

### DETAILED DESCRIPTION OF THE INVENTION

### I. Overview

The present invention provides nanoemulsion universal influenza vaccines formulated for intransal delivery, wherein the vaccines comprise at least one computationally optimized broadly reactive antigen (COBRA) influenza antigen in combination with a nanoemulsion adjuvant. It was surprisingly found that intranasally delivered influenze vaccines, comprising a combination of a nanoemulsion adjuvant and at least one COBRA influenza antigen, produce a dramatic and unexpected protective immune response against more than one subtype or strain of influenza.

The nanoemulsion adjuvant comprises droplets having an average diameter of less than about 1000 nm and (a) an aqueous phase, (b) at least one pharmaceutically acceptable oil, (c) at least one non-ionic surfactant, (d) at least one cationic surfactant; (e) at least one pharmaceutically acceptable organic solvent, and (f) optionally comprising at least one chelating agent. Further, the nanoemulsion vaccine can be formulated into any pharmaceutically acceptable dosage form, such as a liquid dispersion, gel, aerosol, nasal aerosol, or a suspension. The nanoemulsion and/or nanoemulsion vaccine is not systemically toxic to a subject.

The nanoemulsion vaccine effectively prevents, treats or ameliorates influenza infection of a subject. The nanoemulsion vaccine induces a protective immune response in a subject upon administration. In one aspect, a protection response is induced following at least one administration.

The nanoemulsion vaccine can be administered to a subject which has not previously received an influenza vaccine, and the nanoemulsion vaccine can be administered to a subject who had previously received an influenza vaccine. The nanoemulsion vaccine can be given in at least a single administration annually to address seasonal influenza, pandemic flu, or a combination thereof. At least one administration of the nanoemulsion vaccine can be given to provide sustained protection, or more than one administration of the nanoemulsion vaccine can be given to provide sustained protection.

Influenza has been established as a serious human affliction that can cause localized epidemics and global pandemics of acute respiratory infections. Each year the influenza virus is responsible for 20,000 to 40,000 deaths and up to 300,000 hospitalization cases in the U.S. In the pandemic of 1918, it is widely believed that in excess of 40 million people died. The annual influenza epidemics run from November to March in the Northern Hemisphere, and from April to September in the Southern Hemisphere.

The majority of current influenza vaccines have several limitations, including influenza-strain specific immune responses, non-biodegradability, a depot effect, inflammation, and induration at the site of injection, and either a weak, or no cellular immune response. Attempts to increase antibody response by increasing the antigen content per dose have not always resulted in improved immunogenicity. Thus, the present invention, directed to an intranasally-delivered nanoemulsion universal influenza vaccine, satisfies a long felt need in the art.

### Experimental Data

In the experiments described herein (see Examples), it has been demonstrated that mice vaccinated IN with a nanoemulsion universal influenza vaccine comprising a COBRA optimized influenza antigen (H1/H3 rHA) exhibited seroconversion following two or three vaccinations against a panel of H1N1 viruses, including influenza virus A/California/7/09 (H1N1) (CA09), influenza virus A/Brisbane/18/2017 (Bris 18), and influenza virus A/Guangdong-Maonan/SWL1536/2019 (H1N1) (Gaundong-Maonon 19).

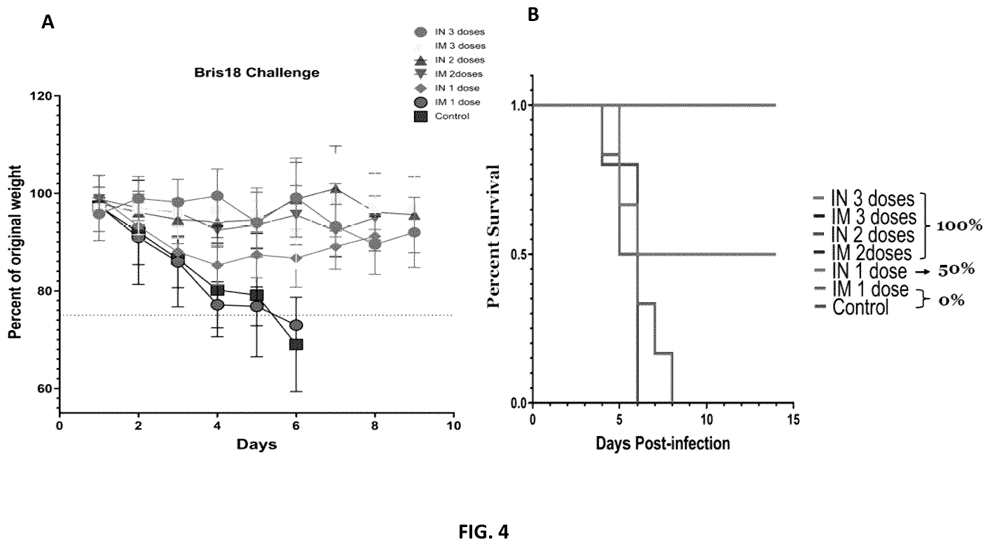

Mice vaccinated two or three times via IN and IM with such COBRA rHA vaccines also exhibited moderate HAI titers against the TX/12 H3N2 virus. In addition, mice vaccinated two or three times via IM or IN administration had no detectable viral titers in lung tissue samples isolated following a viral challenge. Moreover, these mice exhibited no significant weightloss as a result of the viral challenge, highlighting the degree of protection afforded by the vaccines. Further, as detailed in FIG. 4, IN-administration produced approximate weight maintenance, while IM-administered vaccines showed a pronounced weight loss, thus demonstrating that the route of administration has a significant impact on the vaccine response.

Finally, the percent survival was 100% for animals given 2 or 3 doses of IN- or IM-administered vaccine (FIG. 4B). However, at less than 3 vaccine doses, there was a pronounced difference between IN and IM vaccine administration. Specifically, animals given one vaccine dose IM had 0% survival—which was the same as the control group. However, animals delivered one dose IN had a 50% survival rate, which was a dramatic increase as compared to the single dose IM administration group. These results were not anticipated prior to conducting the experiment.

Taken together, these data demonstrate broadly protective systemic and respiratory immune responses against multiple influenza viruses with the IN-administered nanoemulsion vaccine comprising at least one COBRA influenza antigen. These data also illustrate the unexpected result that intranasal delivery of a nanoemulsion universal influenza vaccine comprising a COBRA optimized influenza antigen provides enhanced protection relative to that provided by such a vaccine delivered via IM administration.

The experimental data described herein demonstrate the feasibility of using COBRA-optimized antigens, in combination with an intranasally-delivered nanoemulsion vaccine adjuvant which causes a robust systemic, mucosal systemic, mucosal, and cell-mediated immune response, with added protection at the site of mucosal infection relative to traditional intramuscularly (IM)-delivered vaccines, to successfully generate a universal flu vaccine.

Prior to generation of this data, it was not known if cross-reactive antibodies would be generated using a nanoemulsion COBRA-influenza antigen vaccine. Further, it was not known if IM vs IN would produce a different or preferential immune response and it was also not known if IN or IM vaccination will offer a different degree of protection from the viral challenge.

The nanoemulsion vaccine adjuvant can be combined with the at least one COBRA influenza antigen or the nanoemulsion vaccine adjuvant can be sequentially administered with the at least one COBRA influenza antigen.

The human or animal subject can produce a protective immune response after at least one administration of the nanoemulsion vaccine. In another embodiment, the human or animal subject can produce a protective immune response after at least two administrations of the nanoemulsion vaccine. In one embodiment, the subject undergoes seroconversion after a single administration of the nanoemulsion vaccine, or in another aspect after two administrations of the vaccine. In a further embodiment, the subject is selected from adults, elderly subjects, juvenile subjects, infants, high risk subjects, pregnant women, and immunocompromised subjects.

The immune response of the subject can be measured by determining the titer and/or presence of antibodies against the COBRA-optimized influenza antigen after administration of the nanoemulsion vaccine to evaluate the humoral response to the COBRA-optimized influenza antigen. Seroconversion refers to the development of specific antibodies to an immunogen and may be used to evaluate the presence of a protective immune response. Such antibody-based detection is often measured using Western blotting or enzyme-linked immunosorbent (ELISA) assays or hemagglutination inhibition assays (HAI). Persons of skill in the art would readily select and use appropriate detection methods.

Another method for determining the subject's immune response is to determine the cellular immune response, such as through immunogen-specific cell responses, such as cytotoxic T lymphocytes, or immunogen-specific lymphocyte proliferation assay. Additionally, challenge by the pathogen may be used to determine the immune response, either in the subject, or, more likely, in an animal model. A person of skill in the art would be well versed in the methods of determining the immune response of a subject and the invention is not limited to any particular method.

The nanoemulsion compositions of the invention function as a vaccine adjuvant. Adjuvants serve to: (1) bring the antigen—the substance that stimulates the specific protective immune response—into contact with the immune system and influence the type of immunity produced, as well as the quality of the immune response (magnitude or duration); (2) decrease the toxicity of certain antigens; (3) reduce the amount of antigen needed for a protective response; (4) reduce the number of doses required for protection; (5) provide greater cross-reactivity and protection to heterologous influenza strains; (6) enhance immunity in poorly responding subsets of the population and/or (7) provide solubility to some vaccines components.

Stability: The nanoemulsions vaccines of the disclosure can be stable at about 40° C. and about 75% relative humidity for a time period of at least up to about 2 days, at least up to about 2 weeks, at least up to about 1 month, at least up to about 3 months, at least up to about 6 months, at least up to about 12 months, at least up to about 18 months, at least up to about 2 years, at least up to about 2.5 years, or at least up to about 3 years.

In another embodiment, the nanoemulsion vaccines can be stable at about 25° C. and about 60% relative humidity for a time period of at least up least up to about 2 days, at least up to about 2 weeks, to about 1 month, at least up to about 3 months, at least up to about 6 months, at least up to about 12 months, at least up to about 18 months, at least up to about 2 years, at least up to about 2.5 years, or at least up to about 3 years, at least up to about 3.5 years, at least up to about 4 years, at least up to about 4.5 years, or at least up to about 5 years.

Further, the nanoemulsion vaccines can be stable at about 4° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

The nanoemulsion vaccines can be stable at about −20° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

These stability parameters are also applicable to nanoemulsion adjuvants and/or nanoemulsion vaccines.

### II. COBRA Influenza Antigens

In some embodiments, the nanoemulsion vaccine can comprise at least one computationally optimized broadly reactive (COBRA) antigen. COBRA antigens (e.g., COBRA HA antigens) are able to elicit potent, broadly reactive antibody responses that protect against both vaccine selected and drift variant influenza strains (Allen et al. (2018), PLOS ONE; doi.org/10.1371/journal.pone.0210043). Computational optimization of influenza antigens is described in detail in U.S. Pat. No. 8,883,171, Giles et al., (2012) JID, 2012(205), and Allen et al., (2021) Sci. Rep. 11(4554).

In one embodiment, the nanoemulsion vaccine can comprise about 0.001 μg to about 90 μg of each COBRA optimized influenza antigen, per dose. In a further embodiment, the nanoemulsion vaccine can comprise about 15 μg or less of each COBRA optimized influenza antigen, per dose. In another embodiment, the nanoemulsion vaccine can comprise more than one COBRA optimized influenza antigen, per dose.

Background Regarding Influenza Strains and Subtypes

HA is a viral surface glycoprotein generally comprising approximately 560 amino acids and representing 25% of the total virus protein. It is responsible for adhesion of the viral particle to, and its penetration into, a host cell in the early stages of infection.

Neuraminidase (NA) is a second membrane glycoprotein of the influenza viruses. The presence of viral NA has been shown to be important for generating a multifaceted protective immune response against an infecting virus. For most influenza A viruses, NA is 413 amino acid in length, and is encoded by a gene of 1413 nucleotides.

NA is involved in the destruction of the cellular receptor for the viral HA by cleaving terminal neuraminic acid (also called sialic acid) residues from carbohydrate moieties on the surfaces of infected cells. NA also cleaves sialic acid residues from viral proteins, preventing aggregation of viruses. Using this mechanism, it is hypothesized that NA facilitates release of viral progeny by preventing newly formed viral particles from accumulating along the cell membrane, as well as by promoting transportation of the virus through the mucus present on the mucosal surface. NA is an important antigenic determinant that is subject to antigenic variation.

There are four main strains of influenza virus: A, B, C, and D. Influenza A and B viruses cause the flu season each year, and influenza D is primarily found in non-humans. Influenza A, Influenza B, and Influenza C are distinguished by differences in two major virus surface proteins (Hemagglutinin (HA) and Neuraminidase (NA)). Hemagglutinin (HA) and Neuraminidase (NA) both serve as antigenic determinants found on the surface of the Influenza virus. Influenza A virus is the most common flu virus infecting humans, animals, and birds. Influenza B infection mostly occurred in humans and it does not branch into multiple subtypes. Infection of influenza C virus does not cause any severe symptom in human or mammals and hence it is not well studied.

Influenza A viruses are divided into subtypes on the basis of two proteins on the surface of the virus: hemagglutinin (HA) and neuraminidase (NA). There are 18 known HA subtypes and 11 known NA subtypes for influenza A, but each virus has only one H and one N variant. So, for example, the “H” in “H1N1” refers to hemagglutinin (HA) and “N” in “H1N1” refers to neuraminidase (NA). Influenza viruses have a standard nomenclature that includes virus type; species from which it was isolated (if non-human); location at which it was isolated; isolate number; isolate year; and, for influenza A viruses only, HA and NA subtype. Thus, A/Panama/2007/1999(H3N2) was isolate number 2007 of a human influenza A virus taken in the country of Panama in 1999, and it has an HA subtype 3 and an NA subtype 2. While many genetically distinct subtypes have been found in circulating influenza A viruses, only three HA (H1, H2, and H3) and two NA (N1 and N2) subtypes have caused human epidemics, as defined by sustained, widespread, person-to-person transmission.

Antigenic relatedness within HA facilitates clustering influenza A viruses into two major phylogenetic groups: group 1 (subtypes: H1, H2, H5, H6, H8, H9, H11, H12, H13, H16, H17, and H18) and group 2 (subtypes: H3, H4, H7, H10, H14, and H15). Currently, only influenza A (H1 and H3 subtypes) and B viruses cause seasonal epidemics in humans. However, the perceived threat of highly pathogenic avian influenza viruses (H5N1) and new reports of influenza strains (H7N9, H6N1, and H10N8) crossing over the species barrier and infecting humans necessitate the development of a “universal” influenza vaccine.

Two subtypes of influenza A, H1N1 and H3N2, most commonly infect humans. For each subtype virus, the hemagglutinin gene mutates all the time and hence there are many variants of the same subtype viruses, and therefore the traditional need to change the virus strain for seasonal flu vaccines on an annual bases.

Influenza B mutates at a rate 2-3 times lower than type A and consequently is less genetically diverse, with only one influenza B serotype. Reduced rate of antigenic change, combined with its limited host range (inhibiting cross species antigenic shift), ensures that pandemics of influenza B do not occur.

The influenza A virion is studded with glycoprotein spikes of HA and NA. A number of matrix (M2) ion channels traverse the lipid envelope. The envelope and its three integral membrane proteins HA, NA, and M2, overlay a matrix of M1 protein, which encloses the virion core. Internal to the M1 matrix are found the nuclear export protein (NEP; also called nonstructural protein 2, NS2) and the ribonucleoprotein (RNP) complex, which consists of the viral RNA segments coated with nucleoprotein (NP) and the heterotrimeric RNA-dependent RNA polymerase, composed of two “polymerase basic” and one “polymerase acidic” subunits (PB1, PB2, and PA). The organization of the influenza B virion is similar, with four envelope proteins: HA, NA, and, instead of M2, NB and BM2. Influenza C virions are structurally distinct from those of the A and B viruses, and contain a glycoprotein-studded lipid envelope overlying a protein matrix and the RNP complex. The influenza C viruses have only one major surface glycoprotein, the hemagglutinin-esterase-fusion (HEF) protein, which corresponds functionally to the HA and NA of influenza A and B viruses, and one minor envelope protein, CM2.

There are two known circulating lineages of Influenza B virus based on the antigenic properties of the surface glycoprotein hemagglutinin. The lineages are termed B/Yamagata/16/88-like and B/Victoria/2/87-like viruses. Antigenic variation within the HA and NA antigens of influenzaviruses B has also been analyzed in detail. In contrast to influenzaviruses A, no distinct antigenic subtypes are recognized for members of the species Influenza B virus, however, viruses with antigenically and genetically distinguishable lineages of HA and NA (e.g., the B/Victoria/2/87-like and the B/Yamagata/16/88-like viruses) have co-circulated in humans for over two decades. Influenzaviruses B infect humans and they are designated by their serotype/site of origin/strain designation/year of origin (e.g., B/Victoria/2/87 and B/Yamagata/16/88).

Thus, major outbreaks of influenza are associated with influenza virus type A or B. Influenza A infects birds, humans, swine, horses, seals and dogs. Influenza A is responsible for frequent, usually annual outbreaks or epidemics of varying intensity, and occasional pandemics, whereas influenza B causes outbreaks every two to four years. Influenza B viruses cause the same spectrum of disease as influenza A. However, influenza B viruses do not cause pandemics. Nearly all adults have been infected with influenza C virus, which causes mild upper respiratory tract illness. Lower respiratory tract complications are rare.

The particular structure of the influenza virus genome and function of its viral proteins enable antigenic drift and antigenic shift. These processes result in viruses able to evade the long-term adaptive immune responses in many hosts. Thus, designing an influenza virus vaccine that induces both broad antibody reactivity against co-circulating strains and neutralization across multiple future influenza virus seasons is a pivotal challenge for the development of new influenza vaccines (Allen & Ross, (2021), Sci. Rep., 11(4554)). COBRA HA antigens are capable of eliciting broadly reactive HA-specific antibody responses that can protect against both seasonal and pandemic influenza strains that have undergone genetic drift. These vaccine antigens have also been shown to inhibit viral infection and virus induced pathogenesis in various animal models including mice, ferrets, and non-human primates.

Adapting a broadly reactive influenza virus vaccine design technology, such as COBRA, to industrial settings would be extremely beneficial for both manufacturers and consumers. Aside from potentially reducing the need to update vaccines on an annual basis, technologies like the COBRA methodology provide a promising solution to increase the protection offered by seasonal vaccines against currently co-circulating strains as well as future emerging isolates. Having a broadly reactive vaccine candidate, that has already been antigenically characterized, optimized for production, and is “shelf-ready” would save manufacturers a considerable amount of time, while allowing for year-round production of more doses of vaccine.

Background Regarding COBRA Antigen Optimization

COBRA design methodologies can be focused on developing antigens that are broadly reactive against historical and contemporary influenza vaccine strains.

This COBRA methodology employs multiple rounds of layered consensus sequence alignment building to generate influenza virus vaccine HA antigens that are capable of eliciting broadly reactive HA-specific antibodies that protect against both seasonal and pandemic influenza virus strains. Examples of COBRA influenza techniques are described, for example, in Giles B M, Ross T M., Vaccine, 29:3043-54 (2011) doi:10.1016/j.vaccine.2011.01.100; Giles et al., Clin. Vaccine Immunol., 19:128-139 (2012); Giles et al., J. Infect. Dis., 205:1562-1570 (2012); Crevar et al., Hum. Vaccin. Immunother., 11(3):572-83 (2015); Carter et al., J. of Virology, 90(9):4720-4734 (May, 2016); Wong et al., J. Virol., 91(24):e01581-17 (Nov. 30, 2017); Carter et al., J. Virol., 91:e01217-e01283 (2017); Allen et al., PLoS One, 13(9):e0204284 (Sep. 28, 2018); Bar-Peled et al., Vaccine, 37(41):6022-6029 (Sep. 24, 2019); Sautto et al., J. Immunol., doi:10.4049/jimmunol.1900379 (Dec. 6, 2019); Allen et al., J. Virol., 93:e00946-e1918 (2019); J. Allen and T. Ross, Nature, Scientific Reports, Article No. 4554 (2021); and J. Allen and T. Ross, J. of Virology, www.doi.org/10.1128/jvi.01652-21 (Mar. 15, 2022).

In one embodiment, COBRA influenza antigens are designed using the year-round influenza virus surveillance data, rather than historical influenza virus data. Thus, in one aspect a COBRA antigen can be designed using HA and/or NA viral influenza amino acid sequences collected from any designated time period, e.g., Jan. 1, 2000-Jan. 1, 2022, or any time period inbetween these values. In another aspect, a COBRA antigen can be designed using HA and/or NA viral influenza amino acid sequences from a more recent period, such as for example Jan. 15, 2001-Jan. 1, 2022, or any shortime period within this window. Any desired time period can be used to generate a COBRA antigen based on multiple rounds of layered consensus sequence alignment using HA and/or NA viral influenza amino acid sequences.

For influenza viruses with a wide diversity of HA proteins, developing a broadly reactive influenza vaccine is a challenge. The viral HA protein from H3N2 influenza viruses rapidly evolves via antigenic drift, resulting in frequent emergence of antigenic variant strains that requires updating of the annual influenza vaccine. Antigenic mismatches between the selected strain in the vaccine and cocirculating H3N2 viruses often contribute to reduced vaccine efficacy.

An estimated 20 antigenic clusters have been detected since the H3N2 subtype was introduced into the human population in 1968. To address the need for more broadly reactive influenza vaccines, the methodology of antigen design, termed computationally optimized broadly reactive antigen (COBRA), using multiple rounds of layered consensus building to generate influenza vaccine HA immunogens, has been described. Wong et al., J. Virol., 91(24:e01581-17 (Dec. 15, 2017).

COBRA HA antigens are able to elicit potent, broadly reactive HA-specific antibody responses that protect against both seasonal and novel pandemic influenza virus strains that have undergone genetic drift. Id.

Exemplary COBRA influenza antigens that have been described in the literature, and which can be employed in the described nanoemulsion influenza vaccines, include for example, the following.

TABLE 1

| COBRA | |
|---|---|
| Influenza Antigen | Source |
| T-2, T-4, T-5, T-6, T-7, | Wong et al., J. Virol., 91(24): e01581-17 (Dec. |
| T-8, T-9, T-10, T-11, | 15, 2017) |
| T-12, T-13, T-14, T- | |
| 15, T-16, and T-17 | |
| J4, NG2 | J. Allen and T. Ross, J. of Virology, (Mar. 15, |
| | 2022) |
| | (www.journals.asm.org/doi/abs/10.1128/jvi.01652- |
| | 21) |
| Y2, Y4 | Huang et al., Vaccines, 9(7): 793 (2021) |
| | (doi.org/10.3390/vaccines9070793). |
| NG2 | NG2 was developed from HA sequences spanning |
| | from 2016-2018; Abbadi et al., BioRxiv, |
| | 2022.02.24.481830; doi: |
| | doi.org/10.1101/2022.02.24.481830 |
| J4 | J4 was developed from HA sequences spanning |
| | from 2013-2016. Abbadi et al., BioRxiv, |
| | 2022.02.24.481830; doi: |
| | doi.org/10.1101/2022.02.24.481830 |
| TJ1, TJ2, TJ3, | TJ1-4 were designed utilizing wild-type influenza |
| TJ4, TJ5, TJ6, | virus HA sequences from 2002 to 2007, and TJ5-9 |
| TJ7, TJ8, TJ9 | were generated from HA input sequences that |
| | were in circulation from 2008 to 2015. TJ5 was |
| | developed from HA sequences spanning 2008- |
| | 2012. |
| | J. Allen and T. Ross, Nature, Scientific Reports, |
| | 11: 4554 (2021) |
| | (www.nature.com/articles/s41598-020-79590-7) |
| Z1, Z3, Z5, and Z7 | Reneer et al., J. Virol., 95(2): e01526-20 (Dec. 20, |
| | 2020) |
| Bivalent Mixtures of | J. Allen and T. Ross, J. of Virology, Mar. 15, |
| Y2 + J4 and Y2 + | 2022 (journals.asm.org/doi/abs/10.1128/jvi.01652- |
| NG2 | 21) |

The disclosure is not limited to previously described COBRA antigens, as the present disclosure is directed to the novel discovery that combining a COBRA influenza antigen with a nanoemulsion vaccine adjuvant, which is administered intranasally, results in dramatic and unexpected immune and protective responses.

In one aspect, the nanoemulsion vaccines described herein comprise at least one COBRA antigen which is Y2, J4, and/or NG2. In another aspect of the disclosure, a bivalent COBRA antigen can be present in the nanoemulsion vaccine, such as for example, bivalent mixtures of COBRA H1 and H3 rHA, Y2+J4, or Y2+NG2.

Not all COBRA optimized antigens will result in optimal protection. For example, previously, P1 and X6, two historical COBRA HA vaccines, were designed using the traditional COBRA methodology. These HA antigens elicit broadly reactive antibodies with hemagglutination inhibition (HAI) activity against both historical seasonal and pandemic-like H1N1 influenza viruses isolated from humans and swine. However, COBRA P1 and X6 HA vaccines typically elicit HAI reactive antibodies against H1N1 viruses from 1933 to 2012, but not against H1N1 viruses that circulated after 2012. Therefore, it was critical to generate new COBRA HA vaccines so that they elicit broadly reactive antibodies against currently circulating pandemic-like strains, and also neutralize isolates across multiple future flu seasons.

Previous COBRA design methodologies focus on generating antigens that are broadly reactive against historical and contemporary influenza virus vaccine strains. An emphasis was placed on designing the vaccines using historical influenza isolates, viruses from specific antigenic eras, or past outbreaks. In one aspect of the disclosure, a seasonal-based methodology focuses on current and recent circulating viruses is used to update these broadly reactive HA vaccines to better represent the antigen diversity among currently circulating viruses. Using this new methodology, two promising next generation H1N1 COBRA HA vaccine candidates, Y2 and Y4, were generated to elicit antibody responses against a panel of H1N1 viruses isolated from 1983 to 2021. Each COBRA HA antigen was either expressed as soluble trimerized HA proteins or on virus-like particles (VLPs) as immunogens for testing their efficacy in various strains of mice. Y2 and NG2 were combined with a nanoemulsion adjuvant for evaluation as an improved novel vaccine formulation, as detailed in the Examples below.

Optimized influenza HA and NA polypeptides and influenza are provided herein. The optimized HA and NA polypeptides can be administered to elicit a broadly-reactive immune response against multiple influenza strains.

Disclosed herein is the development of computationally optimized influenza HA and NA proteins that elicit broadly reactive immune responses to influenza virus isolates. The optimized HA protein was developed through a series of HA protein alignments, and subsequent generation of consensus sequences. The final consensus HA amino acid sequence was reverse translated and optimized for expression in mammalian cells. Optimization of the nucleic acid sequence included optimization of the codons for expression in mammalian cells and RNA optimization (such as RNA stability). An exemplary optimized HA protein sequence is set forth herein as SEQ ID NO: 1: MKAILVVLLYTFTTANADTLCIGYHANNSTDTVDTVLEKNVTVTHSVNLLEDK HNGKLCKLRGVAPLHLGKCNIAGWILGNPECESLSTASSWSYIVETSNSDNGTC YPGDFINYEELREQLSSVSSFERFEIFPKTSSWPNHDSNKGVTAACPHAGAKSFY KNLIWLVKKGNSYPKLSQSYINDKGKEVLVLWGIHHPSTTADQQSLYQNADAY VFVGTSRYSKKFKPEIAIRPKVRDQEGRMNYYWTLVEPGDKITFEATGNLVVPR YAFTMERNAGSGIIISDTPVHDCNTTCQTPEGAINTSLPFQNVHPITIGKCPKYVK STKLRLATGLRNVPSIQSRGLFGAIAGFIEGGWTGMVDGWYGYHHQNEQGSGY AADLKSTQNAIDKITNKVNSVIEKMNTQFTAVGKEFNHLEKRIENLNKKVDDGF LDIWTYNAELLVLLENERTLDYHDSNVKNLYEKVRNQLKNNAKEIGNGCFEFY HKCDNTCMESVKNGTYDYPKYSEEAKLNREKIDGVKLESTRIYGSGYIPEAPRD GQAYVRKDGEWVLLSTFLGLNDIFEAQKIEWHEGHHHHHH. Another exemplary optimized HA protein sequence is set forth herein as SEQ ID NO: 2: MKTIIALSYILCLVFAQKIPGNDNSTATLCLGHHAVPNGTIVKTITNDRIEVTNAT ELVQNSSIGEICDSPHQILDGENCTLIDALLGDPQCDGFQNKKWDLFVERSKAYS NCYPYDVPDYASLRSLVASSGTLEFKNESFNWTGVTQNGTSSACIRGSSSSFFSR LNWLTHLNYTYPALNVTMPNNEQFDKLYIWGVHHPGTDKDQIFLYAQSSGRIT VSTKRSQQAVIPNIGSRPRIRDIPSRISIYWTIVKPGDILLINSTGNLIAPRGYFKIRS GKSSIMRSDAPIGKCKSECITPNGSIPNDKPFQNVNRITYGACPRYVKQSTLKLAT GMRNVPEKQTRGIFGAIAGFIENGWEGMVDGWYGFRHQNSEGRGQAADLKST QAAIDQINGKLNRLIGKTNEKFHQIEKEFSEVEGRIQDLEKYVEDTKIDLWSYNA ELLVALENQHTIDLTDSEMNKLFEKTKKQLRENAEDMGNGCFKIYHKCDNACIG SIRNGTYDHNVYRDEALNNRFQIKGVEGYIPEAPRDGQAYVRKDGEWVLLSTFL GSGLNDIFEAQKIEWHEGHHHHHH.

COBRA optimized influenza antigens may contain elements of one or more of the following influenza strains, including, but not limited influenza A virus, influenza B virus or influenza C virus. At present, there are there are 18 different HA subtypes and 11 different NA subtypes of influenza A. Influenza B viruses are not divided into subtypes, but instead are further classified into two lineages: B/Yamagata and B/Victoria. Similar to influenza A viruses, influenza B viruses can then be further classified into specific clades and sub-clades. A COBRA optimized influenza antigen may comprise genetic material from any one or more of these influenza lineages, subtypes or strains.

More specifically, the COBRA optimized influenza antigen may comprise elements of, for example, one or more of.
(1) inactivated influenza virus, a recombinant immunogenic variant of an inactivated influenza virus, or an immunogenic fragment of an inactivated influenza virus;
(2) H5N1, a recombinant immunogenic variant of H5N1, or an immunogenic fragment of H5N1;
(3) H1N1, a recombinant immunogenic variant of H1N1, or an immunogenic fragment of H1N1;
(4) H1N2, a recombinant immunogenic variant of H1N2, or an immunogenic fragment of H1N2;
(5) H3N2, a recombinant immunogenic variant of H3N2, or an immunogenic fragment of H3N2;
(6) H2N2, a recombinant immunogenic variant of H2N2, or an immunogenic fragment of H2N2;
(7) H7N7, a recombinant immunogenic variant of H7N7, or an immunogenic fragment of H7N7;
(8) H9N2, a recombinant immunogenic variant of H9N2, or an immunogenic fragment of H9N2;
(9) H7N2, a recombinant immunogenic variant of H7N2, or an immunogenic fragment of H7N2;
(10) H7N3, a recombinant immunogenic variant of H7N3, or an immunogenic fragment of H7N3;
(11) H10N7, a recombinant immunogenic variant of H10N7, or an immunogenic fragment of H10N7;
(12) H1, a recombinant immunogenic variant of H1, or an immunogenic fragment of H1;
(13) H2, a recombinant immunogenic variant of H2, or an immunogenic fragment of 112;
(14) H3, a recombinant immunogenic variant of H3, or an immunogenic fragment of 113;
(15) H5, a recombinant immunogenic variant of H5, or an immunogenic fragment of 115;
(16) H7, a recombinant immunogenic variant of H7, or an immunogenic fragment of 117;
(17) H9, a recombinant immunogenic variant of H9, or an immunogenic fragment of 119;
(18) N1, a recombinant immunogenic variant of N1, or an immunogenic fragment of N1;
(19) N2, a recombinant immunogenic variant of N2, or an immunogenic fragment of N2;
(20) N3, a recombinant immunogenic variant of N3, or an immunogenic fragment of N3;
(21) N7, a recombinant immunogenic variant of N7, or an immunogenic fragment of N7;
(22) a seasonal influenza strain, a recombinant immunogenic variant of a seasonal influenza strain, or an immunogenic fragment of a seasonal influenza strain;
(23) a pandemic influenza strain, a recombinant immunogenic variant of a pandemic influenza strain, or an immunogenic fragment of a pandemic influenza strain;
(24) an influenza A virus strain, a recombinant immunogenic variant of an influenza A virus strain, or an immunogenic fragment of an influenza A virus strain;
(25) an influenza B virus strain, a recombinant immunogenic variant of an influenza B virus strain, or an immunogenic fragment of an influenza B virus strain;
(26) an influenza C virus strain, a recombinant immunogenic variant of an influenza C virus strain, or an immunogenic fragment of an influenza C virus strain;
(27) A/New Caledonia/20/99 lineage;
(28) A/Fujian/411/2002 lineage;
(29) A/Kumamoto/102/2002 lineage;
(30) A/Wyoming/3/2003 lineage;
(31) A/Wellington/1/2004 lineage;
(32) A/California/7/2004 lineage;
(33) A/New York/55/2004 lineage;
(34) A/Solomon Islands/3/2006 lineage;
(35) A/Wisconsin/67/2005 lineage;
(36) A/Hiroshima/52/2005 lineage;
(37) A/Brisbane/10/2007 lineage;
(38) B/Hong Kong/330/2001 lineage;
(39) B/Shandong/7/97 lineage;
(40) B/Hong Kong/1434/2002 lineage;
(41) B/Brisbane/32/2002 lineage;
(42) B/Shanghai/361/2002 lineage;
(43) B/Jiangsu/10/2003 lineage;
(44) B/Jilin/20/2003 lineage;
(45) B/Malaysia/2506/2004 lineage;
(46) B/Florida/4/2006 lineage;
(47) B/Victoria/2/87 lineage;
(48) B/Yamagata/16/88 lineage;
(49) C/Aichi/1/99 lineage;
(50) C/Sao Paulo/378/82 lineage;
(51) C/Yamagata/26/81 lineage;
(52) C/Aichi/1/81 lineage;
(53) C/Aomori/74 lineage;
(54) C/Mississippi/80 lineage;
(55) any new strain or subtype that may arise due to antigenic drift and/or mutation; and/or
(56) any combination thereof.

In some embodiments, COBRA optimized influenza antigens may comprise elements of one or more of the influenza A, B, and/or C strains identified since 1933.

### III. Nanoemulsion Vaccine Adjuvants

Nanoemulsions are oil-in-water emulsions composed of nanometer sized droplets with surfactant(s) at the oil-water interface. Because of their size, the nanoemulsion droplets are pinocytosed by dendritic cells triggering cell maturation and efficient antigen presentation to the immune system. When combined with a COBRA-optimized influenza antigen, the nanoemulsion vaccine adjuvant elicits and up-modulates strong humoral and cellular T_{H}1-type responses as well as mucosal immunity.

The term “nanoemulsion”, as defined herein, refers to a dispersion or droplet or any other lipid structure. Typical lipid structures contemplated in the invention include, but are not limited to, unilamellar, paucilamellar and multilamellar lipid vesicles, micelles and lamellar phases. Thus, at least a portion of the emulsion may be in the form of lipid structures including, but not limited to, unilamellar, multilamellar, and paucliamellar lipid vesicles, micelles, and lamellar phases.

The nanoemulsions vaccine adjuvant, upon pharmaceutically acceptable administration, are capable of stimulating an immune response to the COBRA-optimized influenza antigen. The at least one COBRA-optimized influenza antigen is typically administered in the composition comprising the nanoemulsion vaccine adjuvant.

In one embodiment, the nanoemulsion vaccine adjuvant comprises droplets having an average diameter of less than about 1000 nm and: (a) an aqueous phase; (b) about 1% oil to about 80% (w/w) pharmaceutically acceptable oil; (c) about 0.1% to about 50% (w/w) organic solvent; (d) about 0.001% to about 10% (w/w) of a non-ionic surfactant; and (e) about 0.001% up to about 5.0% (w/w) of a cationic surfactant.

In one embodiment, the nanoemulsion vaccine adjuvant droplets have an average diameter selected from the group consisting of less than about 1000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, greater than about 50 nm, greater than about 70 nm, greater than about 125 nm, and any combination thereof.

In another embodiment, the droplets have an average diameter size greater than about 125 nm and less than or equal to about 600 nm. In a yet another embodiment, the droplets have an average diameter size greater than about 50 nm or greater than about 70 nm, and less than or equal to about 125 nm.

In an exemplary embodiment, the nanoemulsions comprise droplets of an oily discontinuous phase dispersed in an aqueous continuous phase, such as water or PBS. The nanoemulsions are stable, and do not deteriorate even after long storage periods. Certain nanoemulsions are non-toxic and safe when inhaled or contacted to the skin of a subject.

The present disclosure contemplates that many variations of the described nanoemulsions will be useful in the vaccination methods. To determine if a candidate nanoemulsion is suitable, three criteria are analyzed. Using the methods and standards described herein, candidate emulsions can be easily tested to determine if they are suitable. First, the desired ingredients are prepared using the methods described herein, to determine if a nanoemulsion can be formed. If a nanoemulsion cannot be formed, then the candidate is rejected. Second, the candidate nanoemulsion should form a stable emulsion. A nanoemulsion is stable if it remains in emulsion form for a sufficient period to allow its intended use. For example, for nanoemulsions that are to be stored, shipped, etc., it may be desired that the nanoemulsion remain in emulsion form for months to years. Typical nanoemulsions that are relatively unstable, will lose their form within a day. Third, the candidate nanoemulsion should have efficacy for its intended use. For example, the emulsions of the invention should kill or disable influenza virus to a detectable level, or induce a protective immune response to a detectable level. The nanoemulsion can be provided in many different types of containers and delivery systems. The nanoemulsions of the invention may be incorporated into hydrogel formulations.

Aqueous Phase: The aqueous phase can comprise any type of aqueous phase including, but not limited to, water (e.g., H₂O, distilled water, purified water, water for injection, de-ionized water, tap water) and solutions (e.g., phosphate buffered saline (PBS) solution). In certain embodiments, the aqueous phase comprises water at a pH of about 4 to 10, preferably about 6 to 8. The water can be deionized (hereinafter “DiH₂O”). In some embodiments the aqueous phase comprises phosphate buffered saline (PBS). The aqueous phase may further be sterile and pyrogen free.

Organic solvents: Organic solvents in the nanoemulsion vaccine adjuvants of the disclosure include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one aspect of the invention, the organic solvent is an alcohol chosen from a nonpolar solvent, a polar solvent, a protic solvent, or an aprotic solvent.

Suitable organic solvents for the nanoemulsion vaccine adjuvants include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof. A preferred organic solvent is ethanol.

Oil Phase: The oil in the nanoemulsion vaccine adjuvant can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (Simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof. A preferred oil is soybean oil.

Surfactants: Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference.

Non-ionic surfactants: Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O-(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-100, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of ^{polyoxethyene}. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

In one embodiment, the non-ionic surfactant is present in a concentration of about 0.01% to about 5.0%, or the non-ionic surfactant is present in a concentration of about 0.1% to about 3%.

In one aspect, the non-ionic surfactant can be a polysorbate or a Tween compound. In one aspect, the polysorbate may be polysorbate 80 or polysorbate 20. In another aspect, the non-ionic surfactant may have a concentration of about 0.01% to about 5.0%, or about 0.1% to about 3%.

Cationic surfactants: Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl dimethyl benzyl ammonium chloride, Alkyl didecyl dimethyl ammonium chloride, Alkyl dimethyl benzyl ammonium chloride, Alkyl dimethyl benzyl ammonium chloride (C12-16), Alkyl dimethyl benzyl ammonium chloride (C12-18), Alkyl dimethyl benzyl ammonium chloride, dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis (2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with a particular cationic containing compound.

In one embodiment, the cationic surfactant can be cetylpyridinium chloride (CPC). CPC may have a concentration in the nanoemulsion and/or nanoemulsion vaccine of less than about 5.0% and greater than about 0.001%, or further, may have a concentration of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.001%, greater than about 0.002%, greater than about 0.003%, greater than about 0.004%, greater than about 0.005%, greater than about 0.006%, greater than about 0.007%, greater than about 0.008%, greater than about 0.009%, and greater than about 0.010%. In yet another embodiment of the disclosure, the nanoemulsion vaccine comprises a cationic surfactant present in a concentration of about 0.01% to about 2%.

Additional components of the nanoemulsion vaccine adjuvant: Additional compounds suitable for use in the nanoemulsion vaccine adjuvants include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion vaccine, or the additional compounds can be added to the original mixture to be emulsified. In certain of these embodiments, one or more additional compounds are admixed into an existing nanoemulsion composition immediately prior to its use.

The nanoemulsion vaccine adjuvant may further comprise at least one preservative. Suitable preservatives in the nanoemulsion vaccine adjuvants include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis (p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

The nanoemulsion vaccine adjuvant may further comprise at least one pH adjuster. Suitable pH adjusters in the nanoemulsion vaccine of the invention include, but are not limited to, diethyanolamine, lactic acid, monoethanolamine, triethylanolamine, sodium hydroxide, sodium phosphate, semi-synthetic derivatives thereof, and combinations thereof.

In addition, the nanoemulsion vaccine adjuvant can comprise a chelating agent. In one embodiment of the invention, the chelating agent is present in an amount of about 0.0005% to about 1%. Examples of chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol, and a preferred chelating agent is ethylenediaminetetraacetic acid.

The nanoemulsion vaccine adjuvants can comprise a buffering agent, such as a pharmaceutically acceptable buffering agent. Examples of buffering agents include, but are not limited to, 2-Amino-2-methyl-1,3-propanediol, ≥99.5% (NT), 2-Amino-2-methyl-1-propanol, ≥99.0% (GC), L-(+)-Tartaric acid, ≥99.5% (T), ACES, ≥99.5% (T), ADA, ≥99.0% (T), Acetic acid, ≥99.5% (GC/T), Acetic acid, for luminescence, ≥99.5% (GC/T), Ammonium acetate solution, for molecular biology, ˜5 M in H₂O, Ammonium acetate, for luminescence, ≥99.0% (calc. on dry substance, T), Ammonium bicarbonate, ≥99.5% (T), Ammonium citrate dibasic, ≥99.0% (T), Ammonium formate solution, 10 M in H₂O, Ammonium formate, ≥99.0% (calc. based on dry substance, NT), Ammonium oxalate monohydrate, ≥99.5% (RT), Ammonium phosphate dibasic solution, 2.5 M in H₂O, Ammonium phosphate dibasic, ≥99.0% (T), Ammonium phosphate monobasic solution, 2.5 M in H₂O, Ammonium phosphate monobasic, ≥99.5% (T), Ammonium sodium phosphate dibasic tetrahydrate, ≥99.5% (NT), Ammonium sulfate solution, for molecular biology, 3.2 M in H₂O, Ammonium tartrate dibasic solution, 2 M in H₂O (colorless solution at 20° C.), Ammonium tartrate dibasic, ≥99.5% (T), BES buffered saline, for molecular biology, 2× concentrate, BES, 99.5% (T), BES, for molecular biology, ≥99.5% (T), BICINE buffer Solution, for molecular biology, 1 M in H₂O, BICINE, ≥99.5% (T), BIS-TRIS, ≥99.0% (NT), Bicarbonate buffer solution, ≥0.1 M Na₂CO₃, ≥0.2 M NaHCO₃, Boric acid, ≥99.5% (T), Boric acid, for molecular biology, ≥99.5% (T), CAPS, ≥99.0% (TLC), CHES, ≥99.5% (T), Calcium acetate hydrate, ≥99.0% (calc. on dried material, KT), Calcium carbonate, precipitated, ≥99.0% (KT), Calcium citrate tribasic tetrahydrate, ≥98.0% (calc. on dry substance, KT), Citrate Concentrated Solution, for molecular biology, 1 M in H₂O, Citric acid, anhydrous, ≥99.5% (T), Citric acid, for luminescence, anhydrous, ≥99.5% (T), Diethanolamine, ≥99.5% (GC), EPPS, ≥99.0% (T), Ethylenediaminetetraacetic acid disodium salt dihydrate, for molecular biology, 99.0% (T), Formic acid solution, 1.0 M in H₂O, Gly-Gly-Gly, 99.0% (NT), Gly-Gly, ≥99.5% (NT), Glycine, 99.0% (NT), Glycine, for luminescence, ≥99.0% (NT), Glycine, for molecular biology, ≥99.0% (NT), HEPES buffered saline, for molecular biology, 2× concentrate, HEPES, ≥99.5% (T), HEPES, for molecular biology, ≥99.5% (T), Imidazole buffer Solution, 1 M in H₂O, Imidazole, ≥99.5% (GC), Imidazole, for luminescence, ≥99.5% (GC), Imidazole, for molecular biology, ≥99.5% (GC), Lipoprotein Refolding Buffer, Lithium acetate dihydrate, 99.0% (NT), Lithium citrate tribasic tetrahydrate, ≥99.5% (NT), MES hydrate, ≥99.5% (T), MES monohydrate, for luminescence, ≥99.5% (T), MES solution, for molecular biology, 0.5 M in H₂O, MOPS, ≥99.5% (T), MOPS, for luminescence, ≥99.5% (T), MOPS, for molecular biology, ≥99.5% (T), Magnesium acetate solution, for molecular biology, ˜1 M in H₂O, Magnesium acetate tetrahydrate, 99.0% (KT), Magnesium citrate tribasic nonahydrate, ≥98.0% (calc. based on dry substance, KT), Magnesium formate solution, 0.5 M in H₂O, Magnesium phosphate dibasic trihydrate, ≥98.0% (KT), Neutralization solution for the in-situ hybridization for in-situ hybridization, for molecular biology, Oxalic acid dihydrate, ≥99.5% (RT), PIPES, ≥99.5% (T), PIPES, for molecular biology, ≥99.5% (T), Phosphate buffered saline, solution (autoclaved), Phosphate buffered saline, washing buffer for peroxidase conjugates in Western Blotting, 10× concentrate, Piperazine, anhydrous, ≥99.0% (T), Potassium D-tartrate monobasic, 99.0% (T), Potassium acetate solution, for molecular biology, Potassium acetate solution, for molecular biology, 5 M in H₂O, Potassium acetate solution, for molecular biology, ˜1 M in H₂O, Potassium acetate, ≥99.0% (NT), Potassium acetate, for luminescence, ≥99.0% (NT), Potassium acetate, for molecular biology, 99.0% (NT), Potassium bicarbonate, ≥99.5% (T), Potassium carbonate, anhydrous, ≥99.0% (T), Potassium chloride, ≥99.5% (AT), Potassium citrate monobasic, ≥99.0% (dried material, NT), Potassium citrate tribasic solution, 1 M in H₂O, Potassium formate solution, 14 M in H₂O, Potassium formate, ≥99.5% (NT), Potassium oxalate monohydrate, ≥99.0% (RT), Potassium phosphate dibasic, anhydrous, ≥99.0% (T), Potassium phosphate dibasic, for luminescence, anhydrous, ≥99.0% (T), Potassium phosphate dibasic, for molecular biology, anhydrous, ≥99.0% (T), Potassium phosphate monobasic, anhydrous, ≥99.5% (T), Potassium phosphate monobasic, for molecular biology, anhydrous, ≥99.5% (T), Potassium phosphate tribasic monohydrate, ≥95% (T), Potassium phthalate monobasic, ≥99.5% (T), Potassium sodium tartrate solution, 1.5 M in H₂O, Potassium sodium tartrate tetrahydrate, 99.5% (NT), Potassium tetraborate tetrahydrate, 99.0% (T), Potassium tetraoxalate dihydrate, ≥99.5% (RT), Propionic acid solution, 1.0 M in H₂O, STE buffer solution, for molecular biology, pH 7.8, STET buffer solution, for molecular biology, pH 8.0, Sodium 5,5-diethylbarbiturate, ≥99.5% (NT), Sodium acetate solution, for molecular biology, ˜3 M in H₂O, Sodium acetate trihydrate, ≥99.5% (NT), Sodium acetate, anhydrous, 99.0% (NT), Sodium acetate, for luminescence, anhydrous, 99.0% (NT), Sodium acetate, for molecular biology, anhydrous, ≥99.0% (NT), Sodium bicarbonate, ≥99.5% (T), Sodium bitartrate monohydrate, 99.0% (T), Sodium carbonate decahydrate, ≥99.5% (T), Sodium carbonate, anhydrous, ≥99.5% (calc. on dry substance, T), Sodium citrate monobasic, anhydrous, ≥99.5% (T), Sodium citrate tribasic dihydrate, 99.0% (NT), Sodium citrate tribasic dihydrate, for luminescence, ≥99.0% (NT), Sodium citrate tribasic dihydrate, for molecular biology, ≥99.5% (NT), Sodium formate solution, 8 M in H₂O, Sodium oxalate, ≥99.5% (RT), Sodium phosphate dibasic dihydrate, 99.0% (T), Sodium phosphate dibasic dihydrate, for luminescence, ≥99.0% (T), Sodium phosphate dibasic dihydrate, for molecular biology, 99.0% (T), Sodium phosphate dibasic dodecahydrate, ≥99.0% (T), Sodium phosphate dibasic solution, 0.5 M in H₂O, Sodium phosphate dibasic, anhydrous, ≥99.5% (T), Sodium phosphate dibasic, for molecular biology, ≥99.5% (T), Sodium phosphate monobasic dihydrate, ≥99.0% (T), Sodium phosphate monobasic dihydrate, for molecular biology, 99.0% (T), Sodium phosphate monobasic monohydrate, for molecular biology, ≥99.5% (T), Sodium phosphate monobasic solution, 5 M in H₂O, Sodium pyrophosphate dibasic, ≥99.0% (T), Sodium pyrophosphate tetrabasic decahydrate, ≥99.5% (T), Sodium tartrate dibasic dihydrate, ≥99.0% (NT), Sodium tartrate dibasic solution, 1.5 M in H₂O (colorless solution at 20° C.), Sodium tetraborate decahydrate, ≥99.5% (T), TAPS, ≥99.5% (T), TES, ≥99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate-EDTA buffer solution, for molecular biology, TRIS buffered saline, 10× concentrate, TRIS glycine SDS buffer solution, for electrophoresis, 10× concentrate, TRIS phosphate-EDTA buffer solution, for molecular biology, concentrate, 10× concentrate, Tricine, ≥99.5% (NT), Triethanolamine, ≥99.5% (GC), Triethylamine, ≥99.5% (GC), Triethylammonium acetate buffer, volatile buffer, ˜1.0 M in H₂O, Triethylammonium phosphate solution, volatile buffer, ˜1.0 M in H₂O, Trimethylammonium acetate solution, volatile buffer, ˜1.0 M in H₂O, Trimethylammonium phosphate solution, volatile buffer, ˜1 M in H₂O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, Trizma® acetate, ≥99.0% (NT), Trizma® base, ≥99.8% (T), Trizma® base, ≥99.8% (T), Trizma® base, for luminescence, ≥99.8% (T), Trizma® base, for molecular biology, ≥99.8% (T), Trizma® carbonate, ≥98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solution, for molecular biology, pH 8.0, Trizma® hydrochloride, ≥99.0% (AT), Trizma® hydrochloride, for luminescence, ≥99.0% (AT), Trizma® hydrochloride, for molecular biology, ≥99.0% (AT), and Trizma® maleate, ≥99.5% (NT).

### IV. Pharmaceutical Compositions

The nanoemulsion vaccines may be formulated into pharmaceutical compositions that comprise the nanoemulsion vaccine adjuvant and at least one COBRA-optimized influenza antigen in a therapeutically effective amount and suitable, pharmaceutically-acceptable excipients for pharmaceutically acceptable delivery. Such excipients are well known in the art.

By the phrase “therapeutically effective amount” it is meant any amount of the nanoemulsion vaccine that is effective in preventing, treating or ameliorating influenza. By “protective immune response” it is meant that the immune response associated with prevention, treating, or amelioration of influenza. Complete prevention is not required, though is encompassed by the present disclosure. The immune response can be evaluated using the methods discussed herein or by any method known by a person of skill in the art.

Intranasal administration includes administration via the nose, either with or without concomitant inhalation during administration. Such administration is typically through contact by the composition comprising the nanoemulsion vaccine with the nasal mucosa, nasal turbinates or sinus cavity. Administration by inhalation comprises intranasal administration, or may include oral inhalation. Such administration may also include contact with the oral mucosa, bronchial mucosa, and other epithelia.

The pharmaceutical compositions for administration may be applied in a single administration or in multiple administrations.

An exemplary nanoemulsion adjuvant composition according to the invention is designated “W₈₀5EC” adjuvant. The composition of W₈₀5EC adjuvant is shown in the table below (Table 2). The mean droplet size for the W₈₀5EC adjuvant is ˜400 nm. All of the components of the nanoemulsion are included on the FDA inactive ingredient list for Approved Drug Products.

TABLE 2
W₈₀5EC Formulation

| | W₈₀5EC-Adjuvant |
|---|---|
| Function | Mean Droplet Size ≈ 400 nm |
| Aqueous Diluent | Purified Water, USP |
| Hydrophobic Oil | Soybean Oil, USP (super refined) |
| (Core) | |
| Organic Solvent | Dehydrated Alcohol, USP (anhydrous |
| | ethanol) |
| Surfactant | Polysorbate 80, NF |
| Emulsifying Agent | Cetylpyridinium Chloride, USP |
| Preservative | |

Method of manufacture: The nanoemulsion adjuvants are formed by emulsification of an oil, purified water, nonionic detergent, organic solvent and surfactant, such as a cationic surfactant. An exemplary specific nanoemulsion adjuvant is designated as “60% W₈₀5EC”. The 60% W₈₀5EC-adjuvant is composed of the ingredients shown in Table 3 below: purified water, USP; soybean oil USP; Dehydrated Alcohol, USP [anhydrous ethanol]; Polysorbate 80, NF and cetylpyridinium chloride, USP (CPCAll components of this exemplary nanoemulsion are included on the FDA list of approved inactive ingredients for Approved Drug Products.

TABLE 3
Composition of 60% W₈₀5EC-Adjuvant (w/w %)

| Ingredients | 60% W₈₀5EC |
|---|---|
| Purified Water, USP | 54.10% |
| Soybean Oil, USP | 37.67% |
| Dehydrated Alcohol, USP | 4.04% |
| (anhydrous ethanol) | |
| Polysorbate 80, NF | 3.55% |
| Cetylpyridinium Chloride, USP | 0.64% |

The nanoemulsions can be formed using classic emulsion forming techniques. See e.g., U.S. 2004/0043041. In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the invention employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, herein incorporated by reference in their entireties.

The nanoemulsions can be produced in large quantities and are stable for many months at a broad range of temperatures. The nanoemulsion can have textures ranging from that of a semi-solid cream to that of a thin lotion, to that of a liquid and can be applied topically by any pharmaceutically acceptable method as stated above, e.g., by hand, or nasal drops/spray.

The nanoemulsions can be delivered (e.g., to a subject or customers) in any suitable container. Suitable containers can be used that provide one or more single use or multi-use dosages of the nanoemulsion for the desired application. In some embodiments of the invention, the nanoemulsions are provided in a suspension or liquid form. Such nanoemulsions can be delivered in any suitable container including spray bottles and any suitable pressurized spray device. Such spray bottles may be suitable for delivering the nanoemulsions intranasally or via inhalation.

These nanoemulsion-containing containers can further be packaged with instructions for use to form kits.

The invention is further described by reference to the following examples, which are provided for illustration only. The invention is not limited to the examples, but rather includes all variations that are evident from the teachings provided herein. All publicly available documents referenced herein, including but not limited to U.S. patents, are specifically incorporated by reference.

### V. Definitions

The present invention is described herein using several definitions, as set forth below and throughout the application.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All numerical designations, e.g., mass, temperature, time, and concentration, including ranges, are approximations which are varied (+) or (−) by increments of 1, 5, or 10%. It is to be understood, although not always explicitly stated that all numerical designations are preceded by the term “about.”

The term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term. For example, in some embodiments, it will mean plus or minus 5% of the particular term. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number.

As used in the description of the disclosure and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The term “nanoemulsion,” as used herein, includes dispersions or droplets, as well as other lipid structures that can form as a result of hydrophobic forces that drive apolar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases.

The term “subject” as used herein refers to organisms to be treated by the compositions of the present invention. Such organisms include animals (domesticated animal species, wild animals), and humans.

The term “surfactant” refers to any molecule having both a polar head group, which energetically prefers solvation by water, and a hydrophobic tail which is not well solvated by water. The term “cationic surfactant” refers to a surfactant with a cationic head group. The term “non-ionic surfactant” refers to a surfactant with uncharged head groups.

The terms “Hydrophile-Lipophile Balance Index Number” and “HLB Index Number” refer to an index for correlating the chemical structure of surfactant molecules with their surface activity. The HLB Index Number may be calculated by a variety of empirical formulas as described by Meyers, (Meyers, Surfactant Science and Technology, VCH Publishers Inc., New York, pp. 231-245 [1992]), incorporated herein by reference. As used herein, the HLB Index Number of a surfactant is the HLB Index Number assigned to that surfactant in McCutcheon's Volume 1: Emulsifiers and Detergents North American Edition, 1996 (incorporated herein by reference). The HLB Index Number ranges from 0 to about 70 or more for commercial surfactants. Hydrophilic surfactants with high solubility in water and solubilizing properties are at the high end of the scale, while surfactants with low solubility in water which are good solubilizers of water in oils are at the low end of the scale.

The terms “buffer” or “buffering agents” refer to materials which when added to a solution, cause the solution to resist changes in pH.

The terms “chelator” or “chelating agent” refer to any materials having more than one atom with a lone pair of electrons that are available to bond to a metal ion.

The terms “pharmaceutically acceptable” or “pharmacologically acceptable,” as used herein, refer to compositions that do not substantially produce adverse allergic or adverse immunological reactions when administered to a host (e.g., an animal or a human). Such formulations include any pharmaceutically acceptable dosage form. Examples of such pharmaceutically acceptable dosage forms include, but are not limited to, dips, sprays, seed dressings, stem injections, lyophilized dosage forms, sprays, and mists. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like.

The terms “pharmacologically effective amount” or “therapeutically effective amount” of a composition, aminosterol or agent, as provided herein, refer to a nontoxic but sufficient amount of the composition, aminosterol or agent to provide the desired response. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein. For convenience only, exemplary dosages are provided herein. Those skilled in the art can adjust such amounts in accordance with the methods disclosed herein to treat a specific subject suffering from a specified symptom or disorder. The therapeutically effective amount may vary based on the route of administration and dosage form.

As used herein, the term “intranasal(ly)” refers to application of the compositions of the present invention to the surface of the skin and mucosal cells and tissues of the nasal passages, e.g., nasal mucosa, sinus cavity, nasal turbinates, or other tissues and cells which line the nasal passages.

As used herein, the term “topical(ly)” refers to application of the compositions of the present invention to the surface of the skin and mucosal cells and tissues (e.g., respiratory or nasal mucosa, nasal turbinates).

“Optional” or “optionally” means that the subsequently described circumstance may or may not occur, so that the description includes instances where the circumstance occurs and instances where it does not.

The term “administering” as used herein includes prescribing for administration as well as actually administering and includes physically administering by the subject being treated or by another.

As used herein “subject,” “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

The terms “treatment,” “treating,” or any variation thereof includes reducing, ameliorating, or eliminating (i) one or more specified symptoms and/or (ii) one or more symptoms or effects of a specified disorder. The terms “prevention,” “preventing,” or any variation thereof includes reducing, ameliorating, or eliminating the risk of developing (i) one or more specified symptoms and/or (ii) one or more symptoms or effects of a specified disorder.

### EXAMPLES

### Example 1: Exemplary Vaccine Formulations

The present Examples illustrate exemplary universal influenza nanoemulsion vaccines. The nanoemulsion (NE) composition used for intranasal delivery of the universal influenza vaccine was formulated according to Table 4.

TABLE 4
Nanoemulsion composition (NE01)

| Component | Concentration v/v |
|---|---|
| Water | 84.7% |
| Soybean Oil | 12.6% |
| Ethanol | 1.35% |
| Polysorbate 80 | 1.18% |
| Cetylpyridinium chloride | 0.2% |
| (CPC) | |

A first COBRA-optimized HA consensus antigen, designed Y2, was obtained from Drs. James D. Allen and Ted M. Ross, Center for Vaccines and Immunology, University of Georgia. Y2 is a H1N1 COBRA HA consensus sequence and vaccine antigen generated to elicit antibody responses against a panel of H1N1 viruses isolated from 1983 to 2021. Exemplary preparation of this antigen is also detailed in Huang et al., Vaccines, 9(7):793 (2021) (doi.org/10.3390/vaccines9070793). The full length Y2 HA sequences and multiple sequence alignments are shown in Table 5. The full length Y2 HA amino acid sequence is also set forth in SEQ ID NO: 1.

A second COBRA-optimized HA consensus antigen designed, NG2, was also obtained from Dr. Ted M. Ross. NG2 is a H3 COBRA HA optimized consensus sequence and vaccine antigen. See Abbadi et al., BioRxiv, 2022.02.24.481830; doi: doi.org/10.1101/2022.02.24.481830 and Allen J D and Ross™, JVI, 2022.03.15; e0165221. doi: 10.1128/jvi.01652-21. The full length NG2 HA amino acid sequence is set forth in SEQ ID NO: 2.

TABLE 5
Multiple alignment of all full-length HA sequences,
including Y2 and Y4 (excerpted from Huang et al.)

| | |
|---|---|
| | 1 10 20 30 40 50 60 |
| | \| \| \| \| \| \| \| |
| CA/09 | MKAILVVLLYTFATANADTLCIGYHANNSTDTVDTVLEKNVTVTHSVNLLEDKHNGKLCK |
| Bris/18 | MKAILVVLLYTFTTANADTLCIGYHANNSTDTVDTVLEKNVTVTHSVNLLEDKHNGKLCK |
| Y2 | MKAILVVLLYTFTTANADTLCIGYHANNSTDTVDTVLEKNVTVTHSVNLLEDKHNGKLCK |
| Y4 | MKAILVVLLYTFTTANADTLCIGYHANNSTDTVDTVLEKNVTVTHSVNLLEDKHNGKLCK |
| P1 | MKARLLVLLCALAATDADTICIGYHANNSTDTVDTVLEKNVTVTHSVNLLEDSHNGKLCK |
| X6 | MEARLLVLLCAFAATDADTICIGYHANNSTDTVDTVLEKNVTVTHSVNLLEDSHNGKLCL |
| Bris/07 | MKVKLLVLLCTFTATYADTICIGYHANNSTDTVDTVLEKNVTVTHSVNLLENSHNGKLCL |
| | 61 70 80 90 100 110 120 |
| | \| \| \| \| \| \| \| |
| CA/09 | LRGVAPLHLGKCNIAGWILGNPECESLSTASSWSYIVETPSSDNGTCYPGDFIDYEELRE |
| Bris/18 | LGGVAPLHLGKCNIAGWILGNPECESLSTARSWSYIVETSNSDNGTCYPGDFINYEELRE |
| Y2 | LRGVAPLHLGKCNIAGWILGNPECESLSTASSWSYIVETSNSDNGTCYPGDFINYEELRE |
| Y4 | LRGVAPLHLGKCNIAGWILGNPECESLSTARSWSYIVETSNSDNGTCYPGDFINYEELRE |
| P1 | LKGIAPLQLGKCNIAGWLLGNPECESLLSARSWSYIVETPNSDNGTCYPGDFIDYEELRE |
| X6 | LKGIAPLQLGNCSVAGWILGNPECELLISKESWSYIVETPNPENGTCYPGYFADYEELRE |
| Bris/07 | LKGIAPLQLGNCSVAGWILGNPECELLISKESWSYIVEKPNPENGTCYPGHFADYEELRE |
| | 121 130 140 150 160 170 180 |
| | \| \| \| \| \| \| \| |
| CA/09 | QLSSVSSFERFEIFPKTSSWPNHDSNKGVTAACPHAGAKSFYKNLIWLVKKGNSYPKLSK |
| Bris/18 | QLSSVSSFERFEIFPKTSSWPNHDSNKGVTAACPHAGAKSFYKNLIWLVKKGNSYPKLNQ |
| Y2 | QLSSVSSFERFEIFPKTSSWPNHDSNKGVTAACPHAGAKSFYKNLIWLVKKGNSYPKLSQ |
| Y4 | QLSSVSSFERFEIFPKTSSWPNHDSNKGVTAACPHAGAKSFYKNLIWLVKKGNSYPKLNQ |
| P1 | QLSSVSSFERFEIFPKESSWPNHNTTKGVTAACSHAGAKSFYRNLLWLTKKGGSYPKLSK |
| X6 | QLSSVSSFERFEIFPKESSWPNH-TVKGVSASCSHNGKSSFYRNLLWLTGKNGLYPNLSK |
| Bris/07 | QLSSVSSFERFEIFPKESSWPNH-TVKGVSASCSHNGKSSFYRNLLWLTGKNGLYPNLSK |
| | 180 190 200 210 220 230 240 |
| | \| \| \| \| \| \| \| |
| CA/09 | SYINDKGKEVLVLWGIHHPSTSADQQSLYQNADAYVFVGSSRYSKKFKPEIAIRPKVRDQ |
| Bris/18 | TYINDKGKEVLVLWGIHHPPTTADQQXLYQNADAYVFVGTSRYSKKFKPEIATRPKVRDQ |
| Y2 | SYINDKGKEVLVLWGIHHPSTTADQQSLYQNADAYVFVGTSRYSKKFKPEIAIRPKVRDQ |
| Y4 | TYINDKGKEVLVLWGIHHPSTTADQQSLYQNADAYVFVGTSRYSKKFKPEIATRPKVRDQ |
| P1 | SYVNNKGKEVLVLWGVHHPSTSTDQQSLYQNENAYVSVVSSNYNRRFTPEIAERPKVRGQ |
| X6 | SYANNKEKEVLVLWGVHHPPNIGDQRALYHTENAYVSVVSSHYSRKFTPEIAKRPKVRDQ |
| Bris/07 | SYANNKEKEVLVLWGVHHPPNIGNQKALYHTENAYVSVVSSHYSRKFTPEIAKRPKVRDQ |
| | 241 250 260 270 280 290 300 |
| | \| \| \| \| \| \| \| |
| CA/09 | EGRMNYYWTLVEPGDKTTFEATGNLVVPRYAFAMERNAGSGIIISDTPVHDCNTTCQTFK |
| Bris/18 | EGRMNYYWTLVEPGDKTTFEATGNLVVPRYAFTMERNAGSGIIISDTPVHDCNTTCQTAE |
| Y2 | EGRMNYYWTLVEPGDKITFEATGNLVVPRYAFTMERNAGSGIIISDTPVHDCNTTCQTPE |
| Y4 | EGRMNYYWTLVEPGDKITFEATGNLVVPRYAFTMERNAGSGIIISDTPVHDCNTTCQTPE |
| P1 | AGRMNYYWTLLEPGDTIIFEATGNLIAPWYAFALSRGSGSGIITSNASMHECNTKCQTPQ |
| X6 | EGRINYYWTLLEPGDTIIFEANGNLIAPRYAFALSRGFGSGIITSNAPMDECDAKCQTPQ |
| Bris/07 | EGRINYYWTLLEPGDTIIFEANGNLIAPRYAFALSRGFGSGIINSNAPMDKCDAKCQTPQ |
| | 301 310 320 330 340 350 360 |
| | \| \| \| \| \| \| \| |
| CA/09 | GAINTSLPFQNIHPITIGKCPKYVKSTKLRLATGLRNIPSIQSRGLFGAIAGFIEGGWTG |
| Bris/18 | GAINTSLPFQNVHPVTIGKCPKYVKSTKLRLATGLRNVPSIQSRGLFGAIAGFIEGGWTG |
| Y2 | GAINTSLPFQNVHPITIGKCPKYVKSTKLRLATGLRNVPSIQSRGLFGAIAGFIEGGWTG |
| Y4 | GAINTSLPFQNVHPITIGKCPKYVKSTKLRLATGLRNVPSIQSRGLFGAIAGFIEGGWTG |
| P1 | GAINSSLPFQNIHPITIGECPKYVRSTKLRMVTGLRNIPSIQSRGLFGAIAGFIEGGWTG |
| X6 | GAINSSLPFQNVHPVTIGECPKYVRSAKLRMVTGLRNIPSIQSRGLFGAIAGFIEGGWTG |
| Bris/07 | GAINSSLPFQNVHPVTIGECPKYVRSAKLRMVTGLRNIPSIQSRGLFGAIAGFIEGGWTG |
| | 361 370 380 390 400 410 420 |
| | \| \| \| \| \| \| \| |
| CA/09 | MVDGWYGYHHQNEQGSGYAADLKSTQNAIDEITNKVNSVIEKMNTQFTAVGKEFNHLEKR |
| Bris/18 | MVDGWYGYHHQNEQGSGYAADLKSTQNAIDKITNKVNSVIEKMNTQFTAVGKEFNHLEKR |
| Y2 | MVDGWYGYHHQNEQGSGYAADLKSTQNAIDKITNKVNSVIEKMNTQFTAVGKEFNHLEKR |
| Y4 | MVDGWYGYHHQNEQGSGYAADLKSTQNAIDKITNKVNSVIEKMNTQFTAVGKEFNHLEKR |
| P1 | MIDGWYGYHHQNEQGSGYAADQKSTQNAIDGITNKVNSVIEKMNTQFTAVGKEFNNLEKR |
| X6 | MVDGWYGYHHQNEQGSGYAADQKSTQNAIDGITNKVNSVIEKMNTQFTAVGKEFNKLERR |
| Bris/07 | MVDGWYGYHHQNEQGSGYAADQKSTQNAIDGITNKVNSVIEKMNTQFTAVGKEFNKLERR |
| | 421 430 440 450 460 470 480 |
| | \| \| \| \| \| \| \| |
| CA/09 | IENLNKKVDDGFLDIWTYNAELLVLLENERTLDYHDSNVKNLYEKVRSQLKNNAKEIGNG |
| Bris/18 | IENLNKKVDDGFLDIWTYNAELLVLLENERTLDYHDSNVKNLYEKVRNQLKNNAKEIGNG |
| Y2 | IENLNKKVDDGFLDIWTYNAELLVLLENERTLDYHDSNVKNLYEKVRNQLKNNAKEIGNG |
| Y4 | IENLNKKVDDGFLDIWTYNAELLVLLENERTLDYHDSNVKNLYEKVRNQLKNNAKEIGNG |
| P1 | MENLNKKVDDGFLDIWTYNAELLVLLENERTLDFHDSNVKNLYEKVKSQLRNNAKEIGNG |
| X6 | MENLNKKVDDGFLDIWTYNAELLVLLENERTLDFHDSNVKNLYEKVKSQLKNNAKEIGNG |
| Bris/07 | MENLNKKVDDGFLDIWTYNAELLVLLENERTLDFHDSNVKNLYEKVKSQLKNNAKEIGNG |
| | 481 490 500 510 520 530 540 |
| | \| \| \| \| \| \| \| |
| CA/09 | CFEFYHKCDNTCMESVKNGTYDYPKYSEEAKLNREEIDGVKLESTRIYQILAIYSTVASS |
| Bris/18 | CFEFYHKCDNTCMESVKNGTYDYPKYSEEAKLNREKIDGVKLESTRIYQILAIYSTVASS |
| Y2 | CFEFYHKCDNTCMESVKNGTYDYPKYSEEAKLNREKIDGVKLESTRIYQILAIYSTVASS |
| Y4 | CFEFYHKCDNTCMESVKNGTYDYPKYSEEAKLNREKIDGVKLESTRIYQILAIYSTVASS |
| P1 | CFEFYHKCDNECMESVKNGTYDYPKYSEEAKLNREKIDGVKLESMGVYQILAIYSTVASS |
| X6 | CFEFYHKCNNECMESVKNGTYDYPKYSEEAKLNREKIDGVKLESMGVYQILAIYSTVASS |
| Bris/07 | CFEFYHKCNDECMESVKNGTYDYPKYSEEAKLNREKIDGVKLESMGVYQILAIYSTVASS |
| | 541 550 560 |
| | \| \| \| |
| CA/09 | LVLVVSLGAISFWMCSNGSLQCRICI |
| Bris/18 | LVLVVSLGAISFWMCSNGSLQCRICI |
| Y2 | LVLVVSLGAISFWMCSNGSLQCRICI |
| Y4 | LVLVVSLGAISFWMCSNGSLQCRICI |
| P1 | LVLVVSLGAISFWMCSNGSLQCRICI |
| X6 | LVLVVSLGAISFWMCSNGSLQCRICI |
| Bris/07 | LVLVVSLGAISFWMCSNGSLQCRICI |

The universal influenza vaccine was formulated using NE01 for intranasal administration and Addavax™ for IM administration. Addavax is a squalene-based oil-in-water nano-emulsion with a formulation similar to that of MF59® that has been licensed in Europe for adjuvanted flu vaccines.

Two different antigens were included in the vaccine formulation: a Y2 COBRA HA antigen and a NG2 COBRA antigen, as detailed above and in Table 6 below. The vaccine was constructed as a bivalent COBRA H1/H3 recombinant HA (rHA) vaccine. Two formulations were utilized in the experiments below: 20% Nanoemulsion (NE01) vaccine adjuvant combined with Y2 and NG2 COBRA influenza A antigens for intranasal administration and a 50% Addavax™ vaccine adjuvant combined with Y2 and NG2 COBRA influenza A antigens for intramuscular administration. 3 μg of each COBRA antigen were included in the vaccines. To prepare vaccine, antigens in stabilizing buffer (50 mM Tris, pH 8.0, 150 mM NaCl, 10 mM histidine, 8% sucrose) were mixed with NE01 by pipetting.

This example demonstrates the successful formulation of a nanoemulsion influenza vaccine comprising an COBRA-optimized influenza antigen.

### Example 2: Experimental Design: Dosing and Viral Challenge

The present Example describes an experimental design for evaluating the efficacy of COBRA-optimized influenza vaccines in an animal model.

Briefly, 6 groups of female mice aged 6 to 8 weeks were administered 1, 2, or 3 doses of each of the two vaccine formulations described in Example 1. Three groups of mice received the vaccine formulation as 20% NE01 adjuvanted Y2 and NG2 administered intranasally (IN). Three groups of mice received the vaccine formulation as 50% Addavax adjuvanted Y2 and NG2 administered via intramuscular injection (IM). One group of mice served as a negative control group. Mice in this group were mock-vaccinated three times intranasally using PBS.

In particular, 7 different animal groups (DBA/2J mice) were evaluated: (1) Group 1=20% Nanoemulsion (NE01) vaccine adjuvant combined with Y2 and NG2 COBRA influenza A antigens, with 18 animals and 3 doses of 6 μLg/n are administered IN; (2) Group 2=20% Nanoemulsion (NE01) vaccine adjuvant combined with Y2 and NG2 COBRA influenza A antigens, with 15 animals and 2 doses of 6 μLg/n are administered IN; (3) Group 3=20% Nanoemulsion (NE01) vaccine adjuvant combined with Y2 and NG2 COBRA influenza A antigens, with 12 animals and 1 dose of 6 μL/n are administered IN; (4) 50% Addavax™ vaccine adjuvant combined with Y2 and NG2 COBRA influenza A antigens, with 18 animals and 3 doses of 50 μL, 3 μg each, administered IM; (5) Group 5=50% Addavax™ vaccine adjuvant combined with Y2 and NG2 COBRA influenza A antigens, with 18 animals and 2 doses of 50 μL, 3 μg each, administered IM; (6) Group 6=50% Addavax™ vaccine adjuvant combined with Y2 and NG2 COBRA influenza A antigens, with 18 animals and 1 dose of 50 μL, 3 μg each, administered IM; and (7) Group 7=a Control group of 18 animals mock-vaccinated intranasally with PBS.

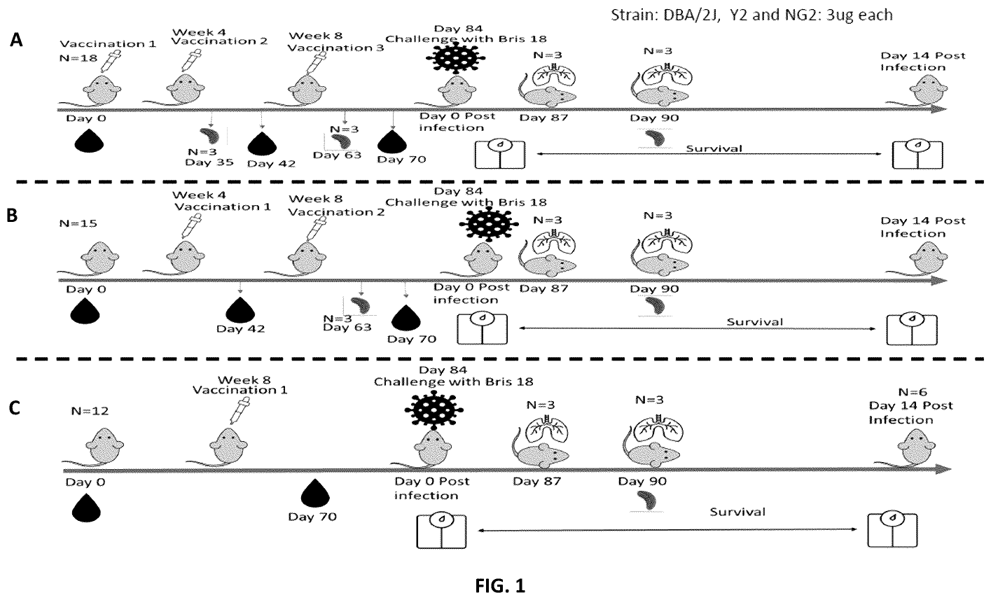

FIG. 1 illustrates the dosing protocol for the three-dose groups (FIG. 1A), the two-dose groups (FIG. 1B), and the one dose groups (FIG. 1C). FIG. 1 also depicts timepoints at which lung tissue samples (indicated by a pictograph of a lung) and blood (serum) samples (indicated by droplets) were obtained from the mice. Table 6 below provides additional details describing the numbers of animals, dose volume, dose number, dosing and sampling schedules, challenge timepoint, and sacrifice timepoint.

TABLE 6
Experimental Design
Total animals: 108

| | | | Dose | | Dosing | Sampling | | |
|---|---|---|---|---|---|---|---|---|
| | | # of | volume | No. of | schedule | schedule | Challenge | Sacrifice |
| Grp | Descriptor | animals | (μL/nare) | Doses | (days) | (days) | on day | on day |
| 1 | 20% NE01 adjuvanted Y2 | 18 | 6 | 3 | 0, 28, 56 | 0, 42, 70 | 84 | 98 |
| | and NG2 | | | | | | | |
| 7 | 20% NE01 adjuvanted Y2 | 15 | 6 | 2 | 28, 56 | 0, 42, 70 | | |
| | and NG2 | | | | | | | |
| 3 | 20% NE01 adjuvanted Y2 | 12 | 6 | 1 | 56 | 0, 70 | | |
| | and NG2 | | | | | | | |
| 4 | 50% Addavax adjuvanted Y2 | 18 | IM (50 ul, | 3 | 0, 28, 56 | 0, 42, 70 | | |
| | and NG2 | | 3 ug each) | | | | | |
| 5 | 50% Addavax adjuvanted Y2 | 15 | IM (50 ul, | 2 | 28, 56 | 0, 42, 70 | | |
| | and NG2 | | 3 ug each) | | | | | |
| 6 | 50% Addavax adjuvanted Y2 | 12 | IM (50 ul, | 1 | 56 | 0, 70 | | |
| | and NG2 | | 3 ug each) | | | | | |
| 7 | Control | 18 | | | | | | |

Results: The results of the experiment revealed that animals treated with a bivalent COBRA H1/H3 rHA nanoemulsion vaccine elicit cross-strain HAI titers. In particular, the present Example illustrates that mice treated with multiple doses of a bivalent H1/H3 rHA antigen (Y2+NG2) combined with a nanoemulsion vaccine adjuvant exhibit significant hemagglutination inhibition titers against multiple influenza viruses.

Serum samples were isolated from treated and control mice, and the serum samples were subjected to a hemagglutination inhibition (HAI) assay. The protocol was adapted from the WHO manual for laboratory influenza surveillance (WHO. 2011. Manual for the laboratory diagnosis and virological surveillance of influenza. WHO, Geneva, Switzerland). To inactivate nonspecific inhibitors, the sera were treated with receptor-destroying enzyme (RDE) (Denka Seiken, Co., Japan) prior to being tested. Briefly, 3 parts RDE was added to 1 part serum and incubated overnight at 37° C. RDE was inactivated by incubating the serum-RDE mixture at 56° C. for approximately 45 min. After the incubation period, 6 parts PBS was added to the RDE-treated serum. RDE-treated serum was 2-fold serially diluted in V-bottom microtiter plates. An equal volume of each virus-like particle (VLP) was adjusted to approximately 8 hemagglutination units (HAU)/25 μl and was added to each well of the V-bottom microtiter plates. The plates were covered and incubated at RT for 20 min before addition of 50 μl of RBCs, which were allowed to settle for 30 min at RT. The HAI titer was determined by the reciprocal dilution of the last well that contained nonagglutinated RBCs. Positive and negative serum controls were included on each plate. At the beginning of the study, mice were negative (HAI titer of <1:10) for antibodies to human and avian H2 HA sequences expressed on VLPs. Seroprotection was defined as an HAI titer of ≥1:40 and seroconversion as a 4-fold increase in titer compared to baseline, as defined by the WHO to evaluate influenza vaccines (WHO. 2011. Manual for the laboratory diagnosis and virological surveillance of influenza. WHO, Geneva, Switzerland.). For our studies, a ≥1:80 HAI titer was also used as a more stringent threshold. Since the mice had an HAI titer of ≤1:10 at the beginning of the study, seroconversion and seroprotection proportions were interchangeable in these studies.

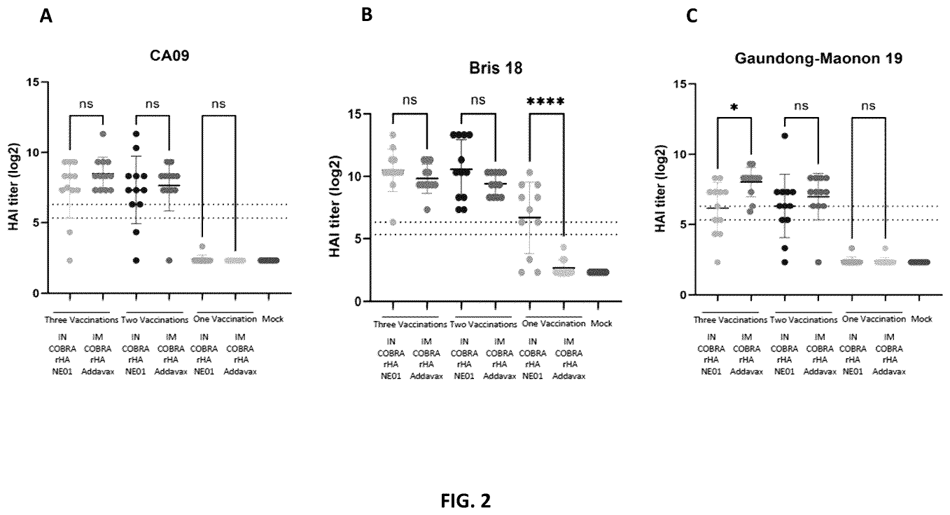

As shown in FIG. 2, mice administered two or three doses of the bivalent COBRA H1/H3 rHA vaccine exhibited significant HAI titers against a panel of H1N1 viruses, including influenza virus A/California/7/09 (H1N1) (CA09) (FIG. 2A), influenza virus B/Brisbane/02/2018 (Bris 18) (FIG. 2B), and influenza virus A/Guangdong-Maonan/SWL1539/2019 (H1N1) (Gaundong-Maonon 19) (FIG. 2C). One dose of the vaccine was not sufficient to generate significant HAI titers. It was highly unexpected that HAI titers against an influenza A and an influenza B virus, and additionally two distinct influenza A viruses, could be generated using a single vaccine formulation.

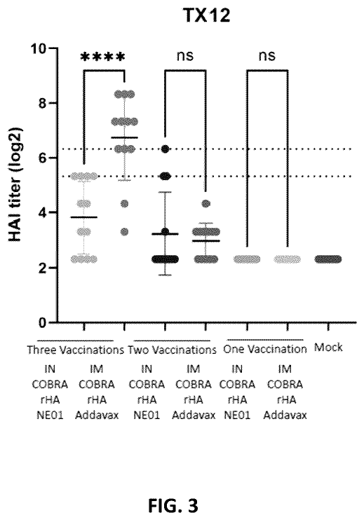

Moreover, as shown in FIG. 3, mice administered two or three doses of the vaccine exhibited significant HAI titers against the influenza A variant, influenza A/Texas/50/2012 (H3N2) (H3N2 influenza virus TX/12), which is a strain of influenza A that can have a more severe impact than other influenza strains and which has also been reported to be drug-resistant (2012-2013 Texas Influenza Surveillance Information, Texas Influenza Summary Report, 2012-2013 Season (Sep. 30, 2012-Sep. 28, 2013 (www.dshs.state.tx.us/IDCU/disease/influenza/surveillance/2012-2013-Texas-Influenza-Surveillance-Information.aspx)).

These data indicate that the bivalent COBRA H1/H3 rHA vaccine elicits a broad cross-reactive and cross-neutralizing antibody response against multiple viral strains, including multiple influenza A strains and additionally influenza B virus. These results are surprising and unexpected, given the long history of challenges in developing an influenza vaccine with the ability to generate immune projection against multiple strains or subtypes of influenza.

### Example 3: Multiple Doses a Bivalent COBRA H1/H3 rHA Vaccine is Protective Following Viral Challenge in Mice

The present Example illustrates that mice treated with multiple doses of a nanoemulsion-adjuvanted, bivalent COBRA H1/H3 rHA vaccine, as described in Example 1, are protected from infection by Influenza strain Bris 18.

Briefly, C57/BL6 mice were administered one, two, or three doses of the bivalent COBRA H1/H3 rHA vaccine according to the dosing protocol described in Table 5 and as described in Example 2. Mice were challenged with Influenza strain Bris 18 starting at day 84 of the experiment. Lung tissue samples were isolated from some of the mice at day 87 and day 90 (FIG. 1). The weight of each mouse was monitored for 14 days following the day the challenge was administered).

As shown in FIG. 4A, mice that received 1 intramuscular dose of the bivalent COBRA H1/H3 rHA vaccine exhibited significant weightloss following the Bris 18 challenge similar to that of control mice. This indicated that 1 intramuscular dose of the vaccine was not protective against infection by Bris 18. Moreover, infection was lethal in these groups, with no mice surviving longer than 8 days post-infection (FIG. 4B). However, mice that received two or three doses of the vaccine did not exhibit significant weight loss following the Bris 18 challenge (FIG. 4A), and 100% of the mice in these groups survived until the end of the experiment (FIG. 4B). Interestingly, mice that were administered a single dose of the vaccine by the IN route also did not exhibit significant weightloss following the challenge (FIG. 4A), and 50% of the mice in this group survived until the end of the experiment (FIG. 4B). This data also demonstrates the surprising result that IN vs IM administration has a striking impact on the resultant immune response.

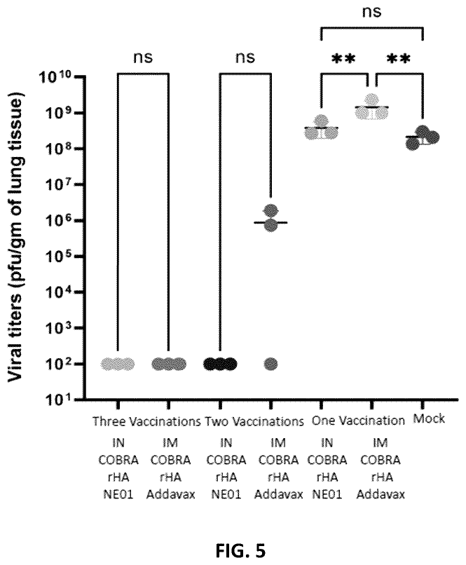

As shown in FIG. 5, mice administered two or three doses of the vaccine by IN or IM routes did not exhibit significant Bris 18 viral titers at Day 3 post-challenge in lung tissue samples following the Bris 18 challenge.

Taken together, these data indicate that two or three doses of the vaccine formulation were protective against infection by Bris 18, and a single dose of the vaccine administered via the IN route was partially protective against the same.

While certain embodiments have been illustrated and described, it should be understood that changes and modifications can be made therein in accordance with ordinary skill in the art without departing from the technology in its broader aspects as defined in the following claims. Thus, it is intended that the present invention cover the modifications and variations of this invention provided they come within the scope of the appended claims and their equivalents.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A nanoemulsion influenza vaccine formulated for intranasal administration and comprising:
(a) at least one computationally optimized broadly reactive antigen (COBRA) influenza antigen;
(b) a nanoemulsion vaccine adjuvant comprising droplets having an average diameter of less than about 1000 nm, and wherein the nanoemulsion comprises:
(i) an aqueous phase;
(ii) at least one pharmaceutically acceptable oil;
(iii) a combination of at least one cationic surfactant and at least one non-ionic surfactant; and
(iv) at least one pharmaceutically organic solvent,
wherein the COBRA influenza antigen is associated with the droplets of the nanoemulsion vaccine adjuvant; and
wherein upon administration the vaccine produces a protective immune response against more than one influenza viral strain.

2. The vaccine of claim 1, wherein upon administration to a subject in need the vaccine induces an immune response in the subject comprising the production of antibodies capable of recognizing:
(a) at least two different hemagglutinins (HA) proteins; or
(b) HA proteins from at least one influenza A virus and at least one influenza B virus; or
(c) HA proteins from at least one influenza C virus; or
(d) HA proteins from at least 2, at least 3, at least 4, at least 5, at least 6, at least 7, at least 8, at least 9, at least 10, at least 11, at least 12, at least 13, at least 14, at least 15, at least 16, at least 17, or 18 different HA proteins from influenza A subtypes; or
(e) HA proteins from influenza B/Yamagata and/or B/Victoria influenza strains; or
(f) HA proteins from all influenza A and/or B strains identified since 1933; or
(g) at least two different neuramidase (NA) proteins; or
(h) NA proteins from at least one influenza A virus and at least one influenza B virus; or
(i) NA proteins from at least 2, at least 3, at least 4, at least 5, at least 6, at least 7, at least 8, at least 9, at least 10, or 11 different subtypes of influenza A;
(j) NA proteins from influenza B/Yamagata and/or B/Victoria influenza strains; or
(k) NA proteins from all influenza A and/or B strains identified since 1933; or
(l) any combination of (a)-(k).

3. The vaccine of claim 1, wherein upon administration to a subject in need the vaccine induces:
(a) an immune response in the subject comprising a Th1 immune response, a Th2 immune response, a Th17 immune response, or any combination thereof, or
(b) a balanced and protective Th1/Th2 immune response in the subject; or
(c) a systemic, mucosal, and cell-mediated immune response in the subject; or
(d) a robust systemic, mucosal, and cell-mediated immune response with minimal inflammation; or
(e) protection at the site of mucosal infection for the subject; or
(f) IL17 and IgA in the subject to provide mucosal protection; or
(g) any combination of (a)-(f).

4. The vaccine of claim 1, wherein:
(a) the COBRA influenza antigen is at least one of Y2, J4, and/or NG2; or
(b) the COBRA influenza antigen comprises recombinant hemagglutinins (rHAs) based upon the amino acid sequence of at least one influenza viral strain; or
(c) the COBRA influenza antigen is based upon one or more amino acid sequences of influenza viruses identified from 1900 to 2022; or
(d) the COBRA HA sequence is based upon one or more HA amino acid sequences from clade 2 H5N1 human infections; or
(e) the COBRA NA sequence is based upon one or more NA amino acid sequences from clade 2 H5N1 human infections; or
(f) any combination of (a)-(e).

5. The vaccine of claim 1, wherein:
(a) the vaccine is a multivalent vaccine; or
(b) the vaccine is a multivalent vaccine comprising a combination of Y2 and J4 COBRA antigens, or a combination of Y2 and NG2 COBRA antigens.

6. The vaccine of claim 1, wherein the COBRA optimized influenza antigen is based upon an amino acid sequence from one or more of the following influenza viral strains:
(a) H1, a recombinant immunogenic variant of H1, or an immunogenic fragment of H1;
(b) H2, a recombinant immunogenic variant of H2, or an immunogenic fragment of H2;
(c) H3, a recombinant immunogenic variant of H3, or an immunogenic fragment of H3;
(d) H5, a recombinant immunogenic variant of H5, or an immunogenic fragment of H5;
(e) H7, a recombinant immunogenic variant of H7, or an immunogenic fragment of H7;
(f) H9, a recombinant immunogenic variant of H9, or an immunogenic fragment of H9;
(g) N1, a recombinant immunogenic variant of N1, or an immunogenic fragment of N1;
(h) N2, a recombinant immunogenic variant of N2, or an immunogenic fragment of N2;
(i) N3, a recombinant immunogenic variant of N3, or an immunogenic fragment of N3;
(j) N7, a recombinant immunogenic variant of N7, or an immunogenic fragment of N7;
(k) a seasonal influenza strain, a recombinant immunogenic variant of a seasonal influenza strain, or an immunogenic fragment of a seasonal influenza strain;
(l) a pandemic influenza strain, a recombinant immunogenic variant of a pandemic influenza strain, or an immunogenic fragment of a pandemic influenza strain;
(m) an influenza A virus strain, a recombinant immunogenic variant of an influenza A virus strain, or an immunogenic fragment of an influenza A virus strain;
(n) an influenza B virus strain, a recombinant immunogenic variant of an influenza B virus strain, or an immunogenic fragment of an influenza B virus strain;
(o) an influenza C virus strain, a recombinant immunogenic variant of an influenza C virus strain, or an immunogenic fragment of an influenza C virus strain;
(p) A/New Caledonia/20/99 lineage;
(q) A/Fujian/411/2002 lineage;
(r) A/Kumamoto/102/2002 lineage;
(s) A/Wyoming/3/2003 lineage;
(t) A/Wellington/1/2004 lineage;
(u) A/California/7/2004 lineage;
(v) A/New York/55/2004 lineage;
(w) A/Solomon Islands/3/2006 lineage;
(x) A/Wisconsin/67/2005 lineage;
(y) A/Hiroshima/52/2005 lineage;
(z) A/Brisbane/10/2007 lineage;
(aa) B/Hong Kong/330/2001 lineage;
(bb) B/Shandong/7/97 lineage;
(cc) B/Hong Kong/1434/2002 lineage;
(dd) B/Brisbane/32/2002 lineage;
(ee) B/Shanghai/361/2002 lineage;
(ff) B/Jiangsu/10/2003 lineage;
(gg) B/Jilin/20/2003 lineage;
(hh) B/Malaysia/2506/2004 lineage;
(ii) B/Florida/4/2006 lineage,
(jj) B/Victoria/2/87 lineage,
(kk) B/Yamagata/16/88 lineage,
(ll) C/Aichi/1/99 lineage,
(mm) C/Sao Paulo/378/82 lineage,
(nn) C/Yamagata/26/81 lineage,
(oo) C/Aichi/1/81 lineage,
(pp) C/Aomori/74 lineage,
(qq) C/Mississippi/80 lineage,
(rr) any new strain or subtype that may arise due to antigenic drift and/or mutation; and
(ss) any combination thereof.

7. The vaccine of claim 1, wherein the nanoemulsion vaccine:
(a) is not systemically toxic to the subject;
(b) produces minimal or no inflammation upon administration;
(c) any combination thereof.

8. The vaccine of claim 1, wherein the nanoemulsion vaccine adjuvant droplets have an average diameter:
(a) selected from the group consisting less than about 1000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, greater than about 50 nm, greater than about 70 nm, greater than about 125 nm, and any combination thereof; or
(b) greater than about 125 nm and less than about 600 nm.

9. The vaccine of claim 1, wherein the organic solvent:
(a) is selected from the group consisting of a C₁-C₁₂ alcohol, diol, triol, dialkyl phosphate, tri-alkyl phosphate, and combinations thereof, or
(b) is is an alcohol selected from the group consisting of a nonpolar solvent, a polar solvent, a protic solvent, an aprotic solvent, semi-synthetic derivatives thereof, and combinations thereof; or
(c) is selected from the group consisting of tri-n-butyl phosphate, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof, or
(d) any combination thereof.

10. The vaccine of claim 1, wherein the oil is:
(a) any cosmetically or pharmaceutically acceptable oil; or
(b) non-volatile; or
(c) selected from the group consisting of animal oil, vegetable oil, natural oil, synthetic oil, hydrocarbon oils, silicone oils, and semi-synthetic derivatives thereof; or
(d) selected from the group consisting of mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (Simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and combinations thereof, or
(d) any combination thereof.

11. The vaccine of claim 1, wherein the non-ionic surfactant:
(a) is selected from the group consisting of nonoxynol-9, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), Brij®35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, Heptaethylene glycol monodecyl ether, Heptaethylene glycol monotetradecyl ether, Heptaethylene glycol monododecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Methyl-6-O-(N-heptylcarbamoyl)-alpha-D-glucopyranoside, Nonaethylene glycol monododecyl ether, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Tergitol Type 15-S-12, Tergitol Type 15-S-30, Tergitol Type 15-S-5, Tergitol Type 15-S-7, Tergitol Type 15-S-9, Tergitol Type NP-10, Tergitol Type NP-4, Tergitol Type NP-40, Tergitol Type NP-7, Tergitol Type NP-9, Tergitol Type TMN-10, Tergitol Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton X-114, Triton X-165, Triton X-305, Triton X-405, Triton X-45, Triton X-705-70, a polysorbate, polysorbate 20, polysorbate 21, polysorbate 40, polysorbate 60, polysorbate 61, polysorbate 65, polysorbate 80, polysorbate 81, polysorbate 85, Tyloxapol, n-Undecyl beta-D-glucopyranoside, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, Poloxamer 182, Dibenzoate, semi-synthetic derivatives thereof, and combinations thereof, or
(b) is a polysorbate; or
(c) is a polysorbate which is polysorbate 20 or polysorbate 80; or
(d) is present at about 0.01% to about 10%, about 0.05% to about 10%, about 0.05% to about 7.0%, about 0.1% to about 7%, about 0.1% to about 3%, or about 0.5% to about 4% (w/w); or
(e) any combination of (a)-(d).

12. The vaccine of claim 1, wherein:
(a) the cationic surfactant is selected from the group consisting of a quarternary ammonium compound, an alkyl trimethyl ammonium chloride compound, a dialkyl dimethyl ammonium chloride compound, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Cetylpyridinium chloride, Dimethyldioctadecylammonium bromide, Dodecylethyldimethylammonium bromide, Dodecyltrimethylammonium bromide, Ethylhexadecyldimethylammonium bromide, Girard's reagent T, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl didecyl dimethyl ammonium chloride, Alkyl dimethyl benzyl ammonium chloride (C12-16), Alkyl dimethyl benzyl ammonium chloride (C12-18), dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis (2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof; or
(b) the cationic surfactant is cetylpyridinium chloride; or
(c) the concentration of the cationic surfactant is less than about 5.0% and greater than about 0.001%, about 0.05% to about 2%, or about 0.01% to about 2% (w/w); or
(d) the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.001%, greater than about 0.002%, greater than about 0.003%, greater than about 0.004%, greater than about 0.005%, greater than about 0.006%, greater than about 0.007%, greater than about 0.008%, greater than about 0.009%, and greater than about 0.010% (w/w); or
(e) any combination of (a)-(d).

13. The vaccine of claim 1, wherein the nanoemulsion vaccine adjuvant comprises:
(a) an aqueous phase;
(b) about 1% oil to about 80% (w/w) of at least one pharmaceutically acceptable oil;
(c) about 0.1% organic solvent to about 50% (w/w) organic solvent;
(d) about 0.001% surfactant to about 10% (w/w) surfactant;
(e) less than about 5.0% and greater than about 0.001% (w/w) of at least one cationic surfactant; and
(f) about 0.01% to about 10% (w/w) of at least one non-ionic surfactant.

14. The vaccine of claim 1 further comprising:
(a) at least one preservative; or
(b) at least one preservative selected from the group consisting of cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, Other suitable preservatives include, but are not limited to, benzyl alcohol, chlorhexidine (bis (p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens), and combinations thereof; or
(c) at least one pH adjuster; or
(d) at least one pH adjuster selected from the group consisting of diethanolamine, lactic acid, monoethanolamine, triethylanolamine, sodium hydroxide, sodium phosphate, semi-synthetic derivatives thereof, and combinations thereof, or
(e) at least one buffer; or
(f) at least one buffer selected from the group consisting of 2-Amino-2-methyl-1,3-propanediol, 2-Amino-2-methyl-1-propanol, L-(+)-Tartaric acid, ACES, ADA, Acetic acid, Ammonium acetate solution, Ammonium bicarbonate, Ammonium citrate dibasic, Ammonium formate, Ammonium oxalate monohydrate, Ammonium phosphate dibasic, Ammonium phosphate monobasic, Ammonium sodium phosphate dibasic tetrahydrate, Ammonium sulfate solution, Ammonium tartrate dibasic, BES buffered saline, BES, BICINE, BIS-TRIS, Bicarbonate buffer solution, Boric acid, CAPS, CHES, Calcium acetate hydrate, Calcium carbonate, Calcium citrate tribasic tetrahydrate, Citrate Concentrated Solution, Citric acid, hydrous, Diethanolamine, EPPS, Ethylenediaminetetraacetic acid disodium salt dihydrate, Formic acid solution, Gly-Gly-Gly, Gly-Gly, Glycine, HEPES, Imidazole, Lipoprotein Refolding Buffer, Lithium acetate dihydrate, Lithium citrate tribasic tetrahydrate, MES hydrate, MES monohydrate, MES solution, MOPS, Magnesium acetate solution, Magnesium acetate tetrahydrate, Magnesium citrate tribasic nonahydrate, Magnesium formate solution, Magnesium phosphate dibasic trihydrate, Oxalic acid dihydrate, PIPES, Phosphate buffered saline, Piperazine, Potassium D-tartrate monobasic, Potassium acetate, Potassium bicarbonate, Potassium carbonate, Potassium chloride, Potassium citrate monobasic, Potassium citrate tribasic solution, Potassium formate, Potassium oxalate monohydrate, Potassium phosphate dibasic, Potassium phosphate dibasic, for molecular biology, anhydrous, Potassium phosphate monobasic, Potassium phosphate monobasic, Potassium phosphate tribasic monohydrate, Potassium phthalate monobasic, Potassium sodium tartrate, Potassium sodium tartrate tetrahydrate, Potassium tetraborate tetrahydrate, Potassium tetraoxalate dihydrate, Propionic acid, STE buffer, STET buffer, Sodium 5,5-diethylbarbiturate, Sodium acetate, Sodium acetate trihydrate, Sodium bicarbonate, Sodium bitartrate monohydrate, Sodium carbonate decahydrate, Sodium carbonate, Sodium citrate monobasic, Sodium citrate tribasic dihydrate, Sodium formate solution, Sodium oxalate, Sodium phosphate dibasic dihydrate, Sodium phosphate dibasic dodecahydrate, Sodium phosphate dibasic solution, Sodium phosphate monobasic dihydrate, Sodium phosphate monobasic monohydrate, Sodium phosphate monobasic solution, Sodium pyrophosphate dibasic, Sodium pyrophosphate tetrabasic decahydrate, Sodium tartrate dibasic dihydrate, Sodium tartrate dibasic solution, Sodium tetraborate decahydrate, TAPS, TES, TM buffer solution, TNT buffer solution, TRIS Glycine buffer, TRIS acetate-EDTA buffer solution, TRIS buffered saline, TRIS glycine SDS buffer solution, TRIS phosphate-EDTA buffer solution, Tricine, Triethanolamine, Triethylamine, Triethylammonium acetate buffer, Triethylammonium phosphate solution, Trimethylammonium acetate solution, Trimethylammonium phosphate solution, Tris-EDTA buffer solution, Trizma® acetate, Trizma® base, Trizma® carbonate, Trizma® hydrochloride, Trizma® maleate, or any combination thereof, or
(g) at least one buffer which is Phosphate Buffered Saline (PBS), wherein the aqueous phase is present in Phosphate Buffered Saline (PBS); or
(h) any combination of (a)-(g).

15. The vaccine of claim 1, wherein the vaccine:
(a) is stable at about 40° C. and about 75% relative humidity for a time period selected from the group consisting of up to about 2 days, up to about 1 week, up to about 2 weeks, up to about 1 month, up to about 3 months, up to about 6 months, up to about 12 months, up to about 18 months, up to about 2 years, up to about 2.5 years, and up to about 3 years; or
(b) is stable at about 25° C. and about 60% relative humidity for a time period selected from the group consisting of up to about 2 days, up to about 1 week, up to about 2 weeks, up to about 1 month, up to about 3 months, up to about 6 months, up to about 12 months, up to about 18 months, up to about 2 years, up to about 2.5 years, up to about 3 years, up to about 3.5 years, up to about 4 years, up to about 4.5 years, and up to about 5 years; or
(c) is stable at about 4° C. for a time period selected from the group consisting of up to about 3 months, up to about 6 months, up to about 12 months, up to about 18 months, up to about 2 years, up to about 2.5 years, up to about 3 years, up to about 3.5 years, up to about 4 years, up to about 4.5 years, up to about 5 years, up to about 5.5 years, up to about 6 years, up to about 6.5 years, and up to about 7 years; or
(d) is stable at about −20° C. for a time period selected from the group consisting of up to about 3 months, up to about 6 months, up to about 12 months, up to about 18 months, up to about 2 years, up to about 2.5 years, up to about 3 years, up to about 3.5 years, up to about 4 years, up to about 4.5 years, up to about 5 years, up to about 5.5 years, up to about 6 years, up to about 6.5 years, and up to about 7 years; or
(e) any combination of (a)-(d).

16. The vaccine of claim 1, wherein the influenza antigen:
(a) is a polypeptide comprising an amino acid sequence that is at least 80%, at least 85%, at least 90%, at least 95%, at least 96%, at least 97%, at least 98%, at least 99%, or 100% identical to SEQ ID NO: 1; or
(b) is a polypeptide comprising an amino acid sequence that is at least 80%, at least 85%, at least 90%, at least 95%, at least 96%, at least 97%, at least 98%, at least 99%, or 100% identical to SEQ ID NO: 2.

17. The vaccine of claim 1, formulated:
(a) for intranasal administration via a nasal spray; or
(b) for intranasal administration via a nasal dropper; or
(c) into a dosage form selected from the group consisting of a liquid dispersion, gel, aerosol, nasal aerosol, and suspensions.

18. The vaccine of claim 1, wherein:
(a) the vaccine is antigen sparing, meaning that per dose the vaccine comprises less antigen as compared to a commercial influenza vaccine, influenza vaccine, or a pandemic influenza vaccine; or
(b) the vaccine comprises about 0.001 μg to about 150 μg of each COBRA optimized influenza antigen; or
(c) the vaccine comprises about 100 μg to about 150 μg COBRA optimized influenza antigen, per dose; or
(d) the vaccine comprises more than one COBRA optimized influenza antigen; or
(e) any combination of (a)-(d).

19. A kit comprising:
(a) the vaccine of claim 1;
(b) a device for nasal administration; and optionally
(c) instructions for administration of the same.

20. A method for inducing an immune response to more than one influenza viral strain in a subject comprising administering to a subject the vaccine according to claim 1, wherein upon administration the vaccine produces a protective immune response against more than one influenza viral strain.

21. A method for inducing an immune response to more than one influenza viral strain in a subject comprising administering sequentially or simultaneously administering to a subject:
(a) at least one computationally optimized broadly reactive antigen (COBRA) influenza antigen;
(b) a nanoemulsion vaccine adjuvant comprising droplets having an average diameter of less than about 1000 nm, and wherein the nanoemulsion comprises:
(i) an aqueous phase;
(ii) at least one pharmaceutically acceptable oil;
(iii) a combination of at least one cationic surfactant and at least one non-ionic surfactant; and
(iv) at least one pharmaceutically organic solvent,
wherein the COBRA influenza antigen is associated with the droplets of the nanoemulsion vaccine adjuvant; and
wherein upon administration the vaccine produces a protective immune response against more than one influenza viral strain.

22. The method of claim 21, wherein:
(i) either (a) or (b) is administered first for sequential administration; or
(ii) the subject produces a protective immune response against more than one influenza viral strain after at least a single administration of the vaccine; or
(iii) the subject undergoes seroconversion after at least a single administration of the vaccine; or
(iv) the vaccine generates a cross-reactive immune and/or antibody response against more than one viral strain; or
(v) the vaccine generates a cross-neutralizing immune and/or antibody response against more than one viral strain; or
(vi) the vaccine generates a broadly reactive, functional immune and/or antibody response against more than one viral strain; or
(vii) any combination of (i)-(vi).

23. The method of claim 21, wherein the vaccine elicits an immune response against two or more HA or NA subtypes or any other immunogenic fragments or recombinant influenza proteins selected from the group consisting of:
(a) H1, a recombinant immunogenic variant of H1, or an immunogenic fragment of H1;
(b) H2, a recombinant immunogenic variant of H2, or an immunogenic fragment of H2;
(c) H3, a recombinant immunogenic variant of H3, or an immunogenic fragment of H3;
(d) H5, a recombinant immunogenic variant of H5, or an immunogenic fragment of H5;
(e) H7, a recombinant immunogenic variant of H7, or an immunogenic fragment of H7;
(f) H9, a recombinant immunogenic variant any of H9, or an immunogenic fragment of H9;
(g) N1, a recombinant immunogenic variant of N1, or an immunogenic fragment of N1;
(h) N2, a recombinant immunogenic variant of N2, or an immunogenic fragment of N2;
(i) N3, a recombinant immunogenic variant of N3, or an immunogenic fragment of N3;
(j) N7, a recombinant immunogenic variant of N7, or an immunogenic fragment of N7;
(k) a seasonal influenza strain, a recombinant immunogenic variant of a seasonal influenza strain, or an immunogenic fragment of a seasonal influenza strain;
(l) a pandemic influenza strain, a recombinant immunogenic variant of a pandemic influenza strain, or an immunogenic fragment of a pandemic influenza strain;
(m) an influenza A virus strain, a recombinant immunogenic variant of an influenza A virus strain, or an immunogenic fragment of an influenza A virus strain;
(n) an influenza B virus strain, a recombinant immunogenic variant of an influenza B virus strain, or an immunogenic fragment of an influenza B virus strain;
(o) an influenza C virus strain, a recombinant immunogenic variant of an influenza C virus strain, or an immunogenic fragment of an influenza C virus strain;
(p) A/New Caledonia/20/99 lineage;
(q) A/Fujian/411/2002 lineage;
(r) A/Kumamoto/102/2002 lineage;
(s) A/Wyoming/3/2003 lineage;
(t) A/Wellington/1/2004 lineage;
(u) A/California/7/2004 lineage;
(v) A/New York/55/2004 lineage;
(w) A/Solomon Islands/3/2006 lineage;
(x) A/Wisconsin/67/2005 lineage;
(y) A/Hiroshima/52/2005 lineage;
(z) A/Brisbane/10/2007 lineage;
(aa) B/Hong Kong/330/2001 lineage;
(bb) B/Shandong/7/97 lineage;
(cc) B/Hong Kong/1434/2002 lineage;
(dd) B/Brisbane/32/2002 lineage;
(ee) B/Shanghai/361/2002 lineage;
(ff) B/Jiangsu/10/2003 lineage;
(gg) B/Jilin/20/2003 lineage;
(hh) B/Malaysia/2506/2004 lineage;
(ii) B/Florida/4/2006 lineage,
(jj) B/Victoria/2/87 lineage,
(kk) B/Yamagata/16/88 lineage,
(ll) C/Aichi/1/99 lineage,
(mm) C/Sao Paulo/378/82 lineage,
(nn) C/Yamagata/26/81 lineage,
(oo) C/Aichi/1/81 lineage,
(pp) C/Aomori/74 lineage,
(qq) C/Mississippi/80 lineage,
(rr) any new strain or subtype that may arise due to antigenic drift and/or mutation; and
(ss) any combination thereof.

24. The method of claim 21, wherein:
(a) the subject is selected from the group consisting of adults, elderly subjects, juvenile subjects, infants, high risk subjects, pregnant women, and immuno-compromised subjects; or
(b) at least a single administration of the vaccine is given at a minimum annually to address seasonal influenza, pandemic influenza, or a combination thereof; or
(c) one or more administrations of the vaccine are given to the subject to provide sustained protection; or
(d) the immune response in the subject comprises the production of antibodies capable of recognizing at least two different HA proteins; or
(e) the immune response in the subject comprises the production of antibodies capable of recognizing HA proteins from all influenza strains identified since 1933; or
(f) the immune response in the subject comprises the production of antibodies capable of recognizing at least two different NA proteins; or
(g) the immune response in the subject comprises the production of antibodies capable of recognizing NA proteins from all influenza strains identified since 1933; or
(h) the immune response in the subject comprises a Th1 immune response, a Th2 immune response, a Th17 immune response, or any combination thereof; or
(i) the immune response in the subject comprises a balanced and protective Th1/Th2 immune response; or
(j) the immune response in the subject comprises a systemic, mucosal, and cell-mediated immune response; or
(k) the immune response in the subject comprises a robust systemic, mucosal, and cell-mediated immune response with minimal inflammation; or
(l) the immune response in the subject comprises protection at the site of mucosal infection; or
(m) the immune response in the subject comprises inducement of IL17 and IgA to provide mucosal protection; or
(n) any combination of (a)-(m).
